(19) United States
(12) Patent Application Publication
(10) Pub. No.: US 2017/0341054 A1
(43) Pub. Date: Nov. 30, 2017

(54) Modification Of Fibers With Nanostructures Using Reactive Dye Chemistry

(71) Applicant: University of South Alabama, Mobile, AL (US)

(72) Inventors: T Grant GLOVER, Spanish Fort, AL (US); Kevin N. WEST, Mobile, AL (US)

(21) Appl. No.: 15/516,637

(22) PCT Filed: Oct. 8, 2015
(86) PCT No.: PCT/US2015/054741

§ 371 Date: Apr. 3, 2017

Related U.S. Application Data
(60) Provisional application No. 62/122,023, filed on Oct. 8, 2014.
(60) Provisional application No. 62/131,607, filed on Mar. 11, 2015.

(51) Int. Cl. B01J 20/22; D06M 13/358; D06M 13/325; B01D 39/08; C07D 251/28; C09B 62/04; B01D 53/02; D06M 23/08; D06M 11/83; B82Y 40/00

(52) U.S. Cl. CPC B01J 20/223; D06M 23/08; D06M 13/358; D06M 13/325; D06M 11/83; B01D 39/08; C07D 251/28; C09B 62/04; B01D 53/02; B01D 2253/25; B82Y 40/00

(57) ABSTRACT

A fiber is provided as a substrate for a functional nanostructure (coated fiber), composed of (a) a fiber substrate; (b) a reactive dye conjugating moiety covalently bound to the fiber substrate; (c) a bonding agent covalently bound to the reactive dye conjugating moiety; and (d) the functional nanostructure bound to the bonding agent. A method of making the coated fiber is also provided, involving the following steps in any order: covalently binding the reactive dye conjugating moiety to the fiber; covalently binding a bonding agent to the reactive dye conjugating moiety; and binding the functional nanostructure to the bonding agent. The nanostructures are tenaciously attached to the fibers, resisting very rough treatments, and can be made using inexpensive and widely available reactive dyes under non-stringent synthesis conditions.

### BACKGROUND

### A. Field of the Disclosure

The present disclosure relates generally to nanotechnological structures. Such structures as well as methods of making and using them are provided.

### B. Background

Functionalizing fibers with tailored chemistry has received interest in both academia and industry, and the results have increased fiber water repellency, fire resistance, fiber strength, and antimicrobial properties. The broad use of natural and synthetic fibers in the form of paper, fabrics, plastic, air filtration media, membranes, and other technologies have resulted in a variety of different methods used to impart chemical functionality to fibers.

Of these methods, particular effort has been placed on synthesizing metal particles by loading cellulose with metal nanoparticle precursors and reacting the precursors to produce a nanometal particle functionalized fabric. Additionally, functionalized cellulose fibers have been synthesized via a layer-by-layer approach where ultrathin organic multilayered films are assembled on a substrate. Still, other methods to modify cellulose have included: the use of carboxyl groups to impart functionality; utilization of sulfur groups to bind silver and other metals; and copper has been embedded for antimicrobial properties. A variety of different types of cellulose have been modified as well, including aerogels and cellulose derived from plant fibers.

Alloys in the form of quantum dots have also been added to both cellulose and nylon fibers. For example, quantum dots have been added to pre-electro spinning polymer solutions to prepare quantum dot fiber composites, and nylon quantum dot hybrid fibers have been prepared via in-situ polymerization. It has also been shown that, water-soluble ZnS quantum dots can be functionalized and utilized in an ink jet printer to impart nanotechnology to the surface of cotton or paper.

In addition to non-porous particles, porous metal organic framework (MOF) structures have been added to fibers using a variety of methods. Specifically, MOF crystals have been grown on cellulose fibers; added to polymer modified fibers; encapsulated in electrospun fibers; immobilized on fibers via solvothermal synthesis methods; developed in layer-by-layer process; and attached using microwave synthesis methods. Of particular interest is the use of atomic layer deposition to grow Cu-BTC MOF crystals on polymer fibers, and the application of MOF materials via ink jet printing onto paper.

However, of all the approaches and techniques used to combine nanostructures with fibers, a broadly applicable approach is absent. Unique synthetic conditions must be developed each time a nanostructure is to be added to a fiber and in some cases, such as atomic layer deposition, sophisticated laboratory equipment is required, which is not effective at an industrial scale. Furthermore, there are no such methods that are readily adaptable to industrial fiber production settings. Currently, there is a need for generic synthetic methods that utilize conditions commonly found in chemical production facilities, such as moderate temperature and pressure requirements, limited vacuum conditions, and methods amenable to roll-to-roll processing technology.

### SUMMARY

The problems described above are addressed in this disclosure by the provision of a fiber coated with a functional nanostructure comprising a reactive dye conjugating moiety; and by the provision of a method of attaching a functional nanostructure to a fiber substrate using a reactive dye conjugating moiety. Reactive dye conjugating moieties bind tenaciously to fibers, do not require highly stringent synthetic conditions, may employ inexpensive commercially available reagents, and are suitable for use in fabric manufacturing facilities.

A general embodiment of the method comprises the following steps in any order: (a) covalently binding the reactive dye conjugating moiety to the fiber substrate; (b) covalently binding a bonding agent to the reactive dye conjugating moiety; and (c) binding the functional nanostructure to the bonding agent.

A general embodiment of the coated fiber comprises: (a) the fiber substrate; (b) a reactive dye conjugating moiety covalently bound to the fiber; (c) a bonding agent covalently bound to the reactive dye conjugating moiety; and (d) the functional nanostructure bound to the bonding agent.

A second general embodiment of the coated fiber is a product of any of the methods above.

A manufactured article is provided, which comprises any of the coated fibers provided above.

The above is a simplified summary in order to provide a basic understanding of some aspects of the claimed subject matter. This summary is not an extensive overview. It is not intended to identify key or critical elements or to delineate the scope of the claimed subject matter. Its sole purpose is to present some concepts in a simplified form as a prelude to the more detailed description that is presented later.

### BRIEF DESCRIPTION OF THE DRAWINGS

FIG. 1 is a schematic illustrating the generic approach utilizing reactive dyes as a platform to attach nanostructures to fibers (1a). A specific example uses the platform to attach gold to nylon fibers. The process proceeds with the attachment of the reactive cyanuric chloride conjugating moiety to nylon (step 1) and then a thiol is added to the cyanuric chloride conjugating moiety via an amine (step 2), and lastly gold, or other metals, are attached to the nylon using the available thiol (step 3) (1b).

FIG. 2 shows cyanuric chloride, which is commonly used to anchor dyes to fabrics, and has been utilized to attach gold to fibers. Shown are field emission scanning electron microscope (FESEM) images of the fabric at 200×, 1000×, and 5000× (a-c) respectively after modification with 40 nm gold particles. Prior to FESEM the gold-modified fibers were washed with water, chloroform, a commercially available hand soap and water, and lastly acetone. The original samples at the same magnifications and without exposure to gold are also shown in images (d-f).

FIG. 3 shows the use of the generic platform technology to attach nanostructures to fibers based on reactive dyes with the attachment of gold to nylon swatches. The color shown on the fabrics is a result of the size of gold particles. The 40 nm sample before and after washing with solvents, water, and soap with water is shown in figures (a) and (b) respectively. Nylon modified with 5 nm particles before and after washing are shown in figures (c) and (d) respectively. The control sample (e) was exposed to 5 nm gold particles and shows no color change indicating effectively no attachment of gold to the fibers.

FIG. 4 illustrates the ability of the metal functionalized fibers to support self-assembled structures, cotton and nylon were functionalized with copper and Cu-BTC MOF was grown on cotton and nylon fabrics. MOF modified cotton is shown in (a-c) at different magnifications and MOF modified nylon is shown in (d). These fabrics show differences in ethane and ethylene adsorption capacity (e), illustrating application of these materials in gas separations. A cotton sample as removed from the MOF reaction solution vial and prior to washing with solvents is shown in (f).

FIG. 5 shows the reactive dye method used to add CdSeS/ZnS quantum dots to nylon fibers. Shown are two fabric samples modified with quantum dots and after being washed with solvents (a and c) and the swatches after being washed with solvents and soap and water (b and d). Also shown is the unmodified fluorescence confocal microscope image of the nylon (e) and the FESEM of the quantum dot modified fabric (f) as well as other quantum dot sizes bound to nylon swatches (e).

FIG. 6 shows fabric samples dyed with gold nanoparticles. For each sample shown, the swatch on the far left is the control, the swatch in the center has been dyed with gold and washed with solvents, and the swatch on the far right has been dyed with gold and then washed with solvents and soap. Nylon dyed with 5 nm gold particles (a), cotton dyed with 20 nm gold (b), cotton dyed with 40 nm gold (c). Sample sizes are provided as approximate lengths to provide scale to the pictures.

FIG. 7 shows samples that have been exposed to gold nanoparticles overnight (a and b) and for 3 days (c). Only minor pink staining is observed indicating limited retention of gold by the cellulose in the absence of RDM chemistry.

FIG. 8 shows samples of nylon and cotton exposed to gold nanoparticles before and after washing. Samples a, c, and e have been exposed to gold for 5 days, 2 days, and 1 day respectively and not washed. Samples b, d, and f have been exposed to gold for 5 days, 2 days, and 1 day respectively and washed with solvents, water, and soap. Samples g, i, and k are cellulose samples exposed to gold for 5 days, 2 days, and 1 day respectively and not washed after exposure to gold. Samples h, j, and l are cellulose samples exposed to gold for 5 days, 2 days, and 1 day respectively and washed with solvent, water, and soap. After washing the fabric gold is not appreciably retained on any of the samples shown in FIG. 3.

FIG. 9 shows fabric samples dyed with different size quantum dots. Cotton samples are shown fluorescing (a), and under ambient lighting (b), similarly nylon fluorescing (c), and under ambient lighting (d). Different size quantum dots are shown fluorescing (e), and under ambient lighting (f). In image (e) nylon unmodified is shown at the far left and continuing to the right are quantum dots at 525, 505, 575, 630, 665 nm.

FIG. 10 shows powder X-ray diffraction data for fabric samples and Cu-BTC control.

FIG. 11 shows micro-breakthrough curves of ammonia on cellulose and nylon modified with Cu-BTC as well as pure Cu-BTC powder.

FIG. 12 shows an image of fibers comprising the cellulose control. Numbers denote areas where EDS spectra were collected.

FIG. 13 shows EDS spectrum from “Area 1” in FIG. 12.

FIG. 14 shows EDS spectrum from “Area 2” in FIG. 12.

FIG. 15 shows an image of fibers comprising the nylon control. Numbers denote areas where EDS spectra were collected.

FIG. 16 shows EDS spectrum from “Area 1” in FIG. 15.

FIG. 17 shows EDS spectrum from “Area 2” in FIG. 15.

FIG. 18 shows an image of cellulose fibers modified with 40 nm gold. Numbers denote areas where EDS spectra were collected.

FIG. 19 shows EDS spectrum from “Area 1” in FIG. 18.

FIG. 20 shows EDS spectrum from “Area 2” in FIG. 18.

FIG. 21 shows an image of nylon modified with 40 nm gold particles. Numbers denote areas where EDS spectra were collected.

FIG. 22 shows EDS spectrum from “Area 1” in FIG. 21.

FIG. 23 shows EDS spectrum from “Area 1” in FIG. 21.

FIG. 24 shows an image of cellulose fibers modified with CdSeS/ZnS alloyed quantum dots. Numbers denote areas where EDS spectra were collected.

FIG. 25 shows EDS spectrum from “Area 1” in FIG. 24.

FIG. 26 shows EDS spectrum from “Area 2” in FIG. 24.

FIG. 27 shows an image of cellulose fibers modified with CdSeS/ZnS alloyed quantum dots. Numbers denote areas where EDS spectra were collected.

FIG. 28 shows EDS spectrum from “Area 1” in FIG. 27.

FIG. 29 shows EDS spectrum from “Area 2” in FIG. 27.

FIG. 30 shows an image of cellulose fibers modified with Cu-BTC MOF. Numbers denote areas where EDS spectra were collected.

FIG. 31 shows EDS spectrum from “Area 1” in FIG. 30.

FIG. 32 shows EDS spectrum from “Area 2” in FIG. 30.

FIG. 33 shows an image of nylon fibers modified with Cu-BTC MOF. Numbers denote areas where EDS spectra were collected.

FIG. 34 shows EDS spectrum from “Area 1” in FIG. 33.

FIG. 35 shows EDS spectrum from “Area 2” in FIG. 33.

FIG. 36 shows cotton control FESEM 200× magnification.

FIG. 37 shows cotton control FESEM 1000× magnification.

FIG. 38 shows cellulose control FESEM 5000× magnification.

FIG. 39 shows nylon modified with quantum dots 1000× magnification showing small aggregate.

FIG. 40 shows nylon modified with quantum dots 5000× magnification focused on aggregated particle.

FIG. 41 shows cellulose modified with quantum dots 200× magnification.

FIG. 42 shows cellulose modified with quantum dots 1000× magnification.

FIG. 43 shows cellulose modified with quantum dots 5000× magnification.

FIG. 44 shows cellulose modified with gold 200× magnification.

FIG. 45 shows cellulose modified with gold 1000× magnification.

FIG. 46 shows cellulose modified with gold 5000× magnification.

### DETAILED DESCRIPTION

### A. Definitions

Unless otherwise defined, all terms (including technical and scientific terms) used herein have the same meaning as are commonly understood by one of ordinary skill in the art of this disclosure. It will be further understood that terms, such as those defined in commonly used dictionaries, should be interpreted as having a meaning that is consistent with their meaning in the context of the specification and should not be interpreted in an idealized or overly formal sense unless expressly so defined herein. Well known functions or constructions may not be described in detail for brevity or clarity.

With reference to the use of the word(s) “comprise” or “comprises” or “comprising” in the foregoing description and/or in the following claims, unless the context requires otherwise, those words are used on the basis and clear understanding that they are to be interpreted inclusively, rather than exclusively, and that each of those words is to be so interpreted in construing the foregoing description and/or the following claims.

The term “consisting essentially of” means that, in addition to the recited elements, what is claimed may also contain other elements (steps, structures, ingredients, components, etc.) that do not adversely affect the operability of what is claimed for its intended purpose as described in this disclosure. This transitional phrase does not encompass embodiments that contain other elements that adversely affect the operability of what is claimed for its intended purpose as described in this disclosure, even if such other elements would be beneficial or neutral with regard to the operability of what is claimed for a purpose other than the ones described in this disclosure.

It will be understood that when a feature or element is referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element, or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments.

The terminology used herein is for the purpose of describing particular embodiments only and is not intended to be limiting. As used herein, the singular forms “a”, “an” and “the” are intended to include the plural forms as well, unless the context clearly indicates otherwise.

The terms “about” or “approximately” mean within a range of reasonable error around a central value. Such reasonable error may for example stem from the precision of an instrument or method used to measure the value. The error could also stem from the precision of a method of making a component of a device. Specific examples of such limits of reasonable error are 20%, 10%, 5%, 2.5%, and 1%. Unless specified otherwise, all numerical values may be approximate.

The terms “first”, “second”, and the like are used herein to describe various features or elements, but these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present disclosure.

### B. Fiber Coated with a Functional Nanostructure

A fiber coated with a functional nanostructure is provided (“coated fiber”), which includes (a) the fiber substrate, (b) a reactive dye conjugating moiety covalently bound to the fiber substrate, (c) a bonding agent covalently bound to the reactive dye conjugating moiety, and (d) the functional nanostructure conjugated to the bonding agent.

The bonding agent may be bound to the functional nanostructure via any type of chemical bond. In some embodiments of the coated fiber, the functional nanostructure is covalently bound to the bonding agent. Such embodiments have the advantage of providing extremely tenacious bonding between the functional nanostructure and the bonding agent.

In some embodiments of the coated fiber, the functional nanostructure is a metal-organic framework (MOF). An MOF refers to a coordination network with organic ligands containing potential voids. In this context a coordination network comprises (1) a coordination compound extending, through repeating coordination entities, in one dimension, but with cross-links between two or more individual chains, loops, or spiro-links; or (2) a coordination compound extending through repeating coordination entities in multiple dimensions. The “coordination compound” is any compound that contains an ion or neutral molecule that is composed of a central atom, such as but not limited to a metal, to which is attached a surrounding array of atoms or groups of atoms, each of which is called a ligand. The term organic ligand is an ion or molecule attached to a coordination compound. In some embodiments of the coated fiber, the MOF is conjugated to an organic linker.

The MOF may be any known in the art. In a specific embodiment of the coated fiber, the MOF is a copper-1,3,5-benzenetricarboxylic acid (Cu-BTC) framework. Cu-BTC frameworks have a variety of useful applications, such as the ability to efficiently scrub contaminants such as ethane, ethylene, and ammonia from gas streams.

The bonding agent is a molecule with at least one reactive functional group, which allows the bonding agent to form a bond with the fiber substrate or the functionalized nanostructure. Some embodiments of the bonding agent comprise at least two reactive functional groups, which allow the bonding agent to form a bond with the fiber substrate and the functionalized nanostructure. In some embodiments of the coated fiber the bonding agent is organic. In further embodiments, the bonding agent is an organic molecule with two or more reactive functional groups, wherein the first functional group covalently binds to the reactive dye conjugating moiety and the second functional group binds to the functional nanostructure.

In some embodiments of the coated fiber, the bonding agent may include a thiol group bound to the functional nanostructure and a primary amine group bound to the reactive dye conjugating moiety. Thiol groups have the advantage of being highly reactive, for example forming strong bonds with metals and other functional groups. Primary amine groups have the advantage of reacting with organohalide compounds, such as certain reactive dye conjugating moieties. Because many fibers lack chlorine atoms, the primary amine allows the bonding agent to selectively react with organohalide reactive dye conjugating moieties, but not with the substrate fiber itself. In some embodiments of the coated fiber, the bonding agent may include a thiol group bound to the functional nanostructure and a primary amine group bound to the reactive dye conjugating moiety.

In some embodiments of the coated fiber, the bonding agent comprises: (1) an SH group; (2) an alkyl group; and (3) one or more functional groups capable of reacting with the reactive dye conjugating moiety. In some embodiments of the coated fiber, the bonding agent may have the formula H₂N—R—SH, in which R is a substituted or unsubstituted alkyl group. In a specific embodiment, R is an unsubstituted alkyl group. In some such embodiments, the R group is no more than 60 carbons in length. In further embodiments, the R group is 1-40 carbons in length, 1-10 carbons in length, 1-6 carbons in length, 1-3 carbons in length; 6 carbons in length; or 3 carbons in length.

In some embodiments of the coated fiber, R is an unbranched and unsubstituted alkyl group, having the formula (CH₂)ₙ. In such embodiments, n indicates the length of the alkyl chain, and may be for example 1-60. In other embodiments of the binding agent n may be 1-40, 1-10, 1-6, 1-3, 6, or 3.

In some embodiments of the coated fiber, the bonding agent is cysteamine (H₂NCH₂CH₂SH) or cysteamine hydrochloride (H₂NCH₂CH₂SH*HCl). These two compounds are widely available commercially, are inexpensive, and pose few hazards to workers or the environment. They also effectively bind some reactive dye conjugating moieties, such as halotriazine moieties.

“Substituted or unsubstituted” or “optionally substituted” means a group such as, for example, but not limited to: alkyl, aryl, heterocyclyl, cycloalkyl, arylalkyl, heteroaryl, heteroarylalkyl, and the like, unless specifically noted otherwise, may be unsubstituted, or may be substituted with 1 or 2 substituents. The term “carbocycle” (or carbocyclyl) as used herein refers to monocyclic, saturated, partially unsaturated or aromatic ring. Carbocycles may be optionally substituted. Non-exclusive examples of carbocycles include cyclopropane, cyclobutane, cyclopentane, cyclohexane, cycloheptane, cyclooctane, cyclopentene, cyclohexene, cyclooctene, cycloheptene, benzyl, naphthalene, anthracene, phenanthrene, biphenyl, and pyrene. The term “hetereocyclyl” or “heterocycle” is a carbocycle group wherein one or more of the atoms forming the ring is a heteroatom that is N, O, or S. The heterocycle may be saturated, partially saturated, or aromatic. Heterocycles may be optionally substituted. Non-exclusive examples of heterocyclyl (or heterocycle) include: piperidyl, 4-morpholyl, 4-piperazinyl, pyrrolidinyl, 1,3-dioxanyl, thiophenyl, furanyl, pyrrolyl, pyrazolyl, pyridinyl, pyrimidinyl, pyranyl, and the like. “Halogenated” or “halo” means fluorine, chloride, bromine or iodine.

Any of a wide variety of functional nanostructures can be included. Such functional nanostructures may include: a nanoparticle of less than about 50 nm diameter, a metal atom, a zero valent metal atom, a single metal atom, a metal ion, a single metal ion, a metalloid atom, a single metalloid atom, a metalloid ion, a single metalloid ion, a quantum dot, a polyoxometalate, a polymer of intrinsic microporosity, a metal-organic framework, a zeolitic imidazolate framework, a graphene oxide framework, a fluorophore, a chromophore, a polypeptide, a protein, an enzyme, a nucleic acid, a carbohydrate, a monosaccharide, a disaccharide, a polysaccharide, cyclodextrin, an organic linker of a metal-organic framework, an antimicrobial compound, an antimicrobial ion, an antimicrobial metal, a quaternary ammonium salt, a metal hydroxide, an amino acid, a monomer, and an antibody.

A nanoparticle of less than about 50 nm diameter has distinct advantages over large particles, specifically that nanoparticles of less than about 50 nm have very large surface areas that enable nanofunctionality, as is known in the art. In some embodiments of the coated fiber, the nanoparticle may include a metal, a metalloid, gold, gold (III), gold chloride, gold (III) chloride, silver, a metal oxide, a metal salt, a magnetic metal, a zeolite, a polymer, silica, graphene, graphite. More specific embodiments of the nanoparticle are a metal atom, a zero valent metal atom, a single metal atom, a metal ion, or a single metal ion selected from the group consisting of: gold, gold (III), silver, mercury, and iron. In an alternative embodiment of the coated fiber, the functional nanostructure is a quantum dot.

Zero valent metals (ZVM) have the advantage of very high thermal conductivity and electrical conductivity. Many ZVM are hard, tough, wear resistant, corrosion resistant, heat resistant, and resistant to deformation at high temperatures. Part of the coated fiber, such as threads or bundles of threads, having the ZVM creates the possibility of enabling the surface metal to act as an electrical or thermal conductor (for example, using zero valent copper or gold). Such a thread with a fiber core and conductive outer layer has a myriad of potential uses in the electronics industry, the textile industry, biomedical device development, and the aerospace field. Such conductive fibers would have significant strength, high conductivity and much lower mass than bulk metal wires used in power transmission and signal transmission.

The reactive dye conjugating moiety is a group from a reactive dye that functionalizes the dye for binding to the fiber. A reactive dye comprises a chromophore and a structure with a reactive group, which structure is referred to in this disclosure as the conjugating moiety. The conjugating moiety reacts with the fiber to form a covalent bond, resulting in very colorfast dying of the fiber. Various reactive dyes and their conjugating groups are known. Any known reactive dye conjugating moiety may be used. In some embodiments of the coated fiber, the reactive dye conjugating moiety may include a reactive binding group selected from the group consisting of: chloride, fluoride, thiol, sulfide, sulfone, and amide. In some embodiments of the coated fiber, the reactive dye conjugating moiety may include at least two reactive binding groups, each independently selected from the group consisting of: chloride, fluoride, thiol, sulfide, sulfone, and amide. In some embodiments of the coated fiber, the reactive dye conjugating moiety is a halorotriazine moiety. In some embodiments of the coated fiber, the reactive dye conjugating moiety is a mono-, di-, or trichlorotriazine moiety. In further embodiments of the coated fiber, the reactive dye conjugating moiety is selected from the group consisting of: cyanuric chloride, monochlorotriazine, monofluorochlorotriazine, dichlorotriazine, monofluorodichlorotriazine, and difluoromonochlorotriazine. In a specific embodiment of the coated fiber, the reactive dye conjugating moiety is a 2,4,6-trichloro-1,3,5-triazine(cyanuric chloride) moiety. The halotriazines have the advantage of excellent fiber binding chemistry in reactive dyes. In alternative embodiments of the coated fiber, the reactive dye conjugating moiety is a vinyl sulfone moiety. Other examples of the reactive dye conjugating moiety include haloquinoxaline moieties and halo pyrimidine moieties.

In some embodiments of the coated fiber, the functional nanostructure is a gold nanoparticle that may further include a functionalized thiol bound to gold in the nanostructure. In some embodiments of the coated fiber, the functional nanostructure that is a gold nanoparticle, a gold atom, gold(III) chloride, or gold (III) chloride hydrate, may further include a functionalized thiol compound bound to the gold nanoparticle. In such embodiments the functional nanostructure may serve as a general substrate for the addition of further groups to the coated fiber.

In some embodiments of the coated fiber, the first functional nanostructure may be conjugated to a second functional nanostructure via a functionalized thiol. As stated above, the first functional nanostructure may be a gold nanoparticle that may further include a functionalized thiol compound bound to a second functional nanostructure. In some embodiments of the coated fiber, the first functional nanostructure is a gold nanoparticle that may further include a functionalized thiol compound bound to a second functional nanostructure, wherein that second functional nanostructure is a gold nanoparticle.

The second functional nanostructure may be any known in the art. In some embodiments of the coated fiber, the functionalized thiol may be conjugated to a second functional nanostructure selected from the group consisting of: a self-assembled monolayer forming compound, gold, silver, metal nanoparticles, bulk metal particles, metal salts, metalloids, metal alloys, quantum dots, adsorbents, catalysts, metal oxides, magnetic nanoparticles, polyoxometalates (POMS), polymers, polymers of intrinsic microporosity (PIMS), metal organic frameworks (MOFs), an organic linker of a MOF, zeolites, covalent organic frameworks (COFs), zeolites, zeolitic imidazolate frameworks (ZIFs), graphene oxide frameworks (GOFs), colloidal particles, silicas, carbons, graphene, graphite, antibiotics, antibodies, fluorescent molecules, enzymes, and proteins.

Any of various fibers may serve as the substrate. In some embodiments of the coated fiber, the fiber substrate is a synthetic fiber, a vegetable fiber, or an animal fiber. Synthetic fibers include nylon, modacrylic, olefin, acrylic, polyester, carbon fiber, rayon, vinyon, saran, spandex, vinalon, araminds, modal, dyneema, specrtra, polybenzimidazole, sulfar, lyocell, PLA, M-5, orion, zylon, vectran, derclon, glass fibers, glass wool, glass reinforced plastics, rayon, diacetate, triacetate, polyamide, aliphatic polyimide, polyphthalamide, and an aramide. Such synthetics are commercially available under tradenames such as nylon, technyl, rilsan, rilsamid, trogamid, amodel, Kevlar, Nomex, Teijinconex, Twaron, Technora, Kermel, and Spectra. In further embodiments of the coated fiber, the fiber substrate is a vegetable fiber. Vegetable fibers include cellulosic fibers, cotton, hemp, jute, flax, ramie, sisal, bagasse, pina, esparto, Indian hemp, hoopvine, kenaf, linden bast, nettle bast, papyrus, Manila hemp, bowstring hemp, henequen, phormium, yucca, coir, kapok, milkweed, luffa, wood fiber, or bamboo fiber. In further embodiments of the coated fiber, the fiber substrate is an animal fiber. The animal fiber may be, for example proteinaceous, silkworm silk, spider silk, sinew, catgut, wool, sea silk, hair, cashmere wool, mohair, angora, sheep pelt, rabbit pelt, mink pelt, fox pelt, beaver pelt, angora, bison, qiviut, horsehair, chiengora, alpaca wool, vicuña wool, merino wool, yak down, camel down, guanaco wool, llama wool, or chinchilla.

Some embodiments of the coated fiber display extremely durable attachment of the functionalized nanostructure to the fiber substrate. In some embodiments of the coated fiber, no significant loss of the functional nanostructure occurs upon vigorous washing. In some embodiments of the coated fiber, no significant loss of the functional nanostructure occurs upon vigorous washing with one or more of: chloroform, water, soap and water, tetrahydrofuran, hexane, and acetone. In some embodiments of the coated fiber, no significant loss of the functional nanostructure occurs upon vigorous washing with all of: chloroform, water, soap and water, tetrahydrofuran, hexane, and acetone. Although the act of “vigorous washing” is well understood by those in the art, where necessary to definitely establish the meaning of such term, the vigorous washing may be construed to be the washing process described below, in the “Materials and Methods” section of the Working Example In various embodiments of the coated fiber, the vigorous washing described above results no significant loss, that is less than 25% loss of the functional nanostructure. In further embodiments the not significant loss is less than 20%, 15%, 10%, 5%, and 1%. In a specific embodiment the loss is about 0%. In still further embodiments, no loss of the functionalized nanostructure to the fiber substrate can be detected by unassisted visual observation.

### C. Method of Attaching a Functional Nanostructure to a Fiber

A method of attaching a functional nanostructure to a fiber substrate using a reactive dye is provided, including the following steps in any order: (a) covalently binding the reactive dye conjugating moiety to the fiber substrate, (b) covalently binding a bonding agent to the reactive dye conjugating moiety, and (c) binding the functional nanostructure to the bonding agent. A specific embodiment of the method includes performing step (a) prior to steps (b) and (c), and then performing step (b) prior to step (c). In some embodiments, step (c) may include covalently binding the functional nanostructure to the bonding agent. Such embodiments have the advantage of providing a durable bond between the functional nanostructure and the bonding agent.

In some embodiments, the functional nanostructure is a metal-organic framework (MOF). In some such embodiments, step (c) may include binding a metal ion to the bonding agent. In further embodiments in which the functional nanostructure is a MOF, the method may include conjugating the metal ion to an organic linker.

The reactive dye conjugating moieties, bonding agents, and functional nanostructures used in the method may be any of the reactive dye conjugating moieties, bonding agents, and functional nanostructures disclosed as suitable for use in the coated fiber above.

In embodiments of the method in which the functional nanostructure comprises a metal atom or ion, the method may further comprise reducing the metal atom or ion to a zero valent state. The atom or ion may be reduced chemically, electrochemically or thermally to yield a zero valent metal atom that remains attached to the surface through the bonding agent and the reactive dye conjugation moiety. In some embodiments the method comprises reducing an atom or ion of gold; however, any metal atom or ion that can be attached would also be reducible by these means (e.g. Cu²⁺, Cu⁺, Ag⁺, Fe²⁺, Fe³⁺, Zn²⁺, Ti⁴⁺, Pd²⁺, Pt²⁺, Zr⁴⁺, Al³⁺, etc.). Such ZVM coatings has numerous advantages and uses as described above.

A fiber coated with a functional nanostructure is provided that is the product of any of the processes disclosed above. In some embodiments of the coated fiber, no significant loss of the functional nanostructure occurs upon vigorous washing. In some embodiments of the coated fiber, no significant loss of the functional nanostructure occurs upon vigorous washing with one or more of: chloroform, water, soap and water, tetrahydrofuran, hexane, and acetone. In some embodiments of the coated fiber, no significant loss of the functional nanostructure occurs upon vigorous washing with all of: chloroform, water, soap and water, tetrahydrofuran, hexane, and acetone. In various embodiments of the clothing or textile, the vigorous washing described above results in less than 25% loss of the functional nanostructure. In further embodiments the loss is less than 20%, 15%, 10%, 5%, and 1%. In a specific embodiment the loss is about 0%.

### D. Article Comprising a Fiber Bound to a Functional Nanostructure

A manufactured article is provided, comprising any of the coated fibers disclosed above. Such article may include a wooden article; in some embodiments of the wooden article, the fiber substrate is a wood fiber. Other examples of the article include a paper article, and a textile (which may be a woven or knit textile). In embodiments of the article that are a textile, the textile may be for example cambric, chino, corduroy, denim, seersucker, or terrycloth.

The article may also be an item of clothing that includes the textile. Some embodiments of the clothing or textile display extremely durable attachment of the functionalized nanostructure to the fiber substrate. In some embodiments of clothing or textile, no significant loss of the functional nanostructure occurs upon vigorous washing. In some embodiments of clothing or textile, no significant loss of the functional nanostructure occurs upon vigorous washing with one or more of: chloroform, water, soap and water, tetrahydrofuran, hexane, and acetone. In some embodiments of clothing or textile, no significant loss of the functional nanostructure occurs upon vigorous washing with all of: chloroform, water, soap and water, tetrahydrofuran, hexane, and acetone. Although the act of “vigorous washing” is well understood by those in the art, where necessary to definitely establish the meaning of such term, the vigorous washing may be construed to involve . . . . In various embodiments of the clothing or textile, the vigorous washing described above results in less than 25% loss of the functional nanostructure. In further embodiments the loss is less than 20%, 15%, 10%, 5%, and 1%. In a specific embodiment the loss is about 0%.

### E. Working Examples

Therefore, the purpose of the examples below is to demonstrate that reactive dye coordinating moieties, such as but not limited to cyanuric chloride, used commercially to add color to fabrics and fibers, can also be used as a generic platform to attach functional nanostructures to substrates. The example utilizes fibers as a substrate and cyanuric chloride as a reactive dye conjugating moiety. Several functional groups bound to the substrate are considered and the growth of a monolayer after functionalizing a substrate is demonstrated.

The utilization of reactive dye chemistry as a method to functionalize fibers provides a robust and cost-effective platform to build or incorporate nanostructures into commercially available fiber products. A general platform approach is illustrated in FIG. 1a showing the attachment of a generic nanostructure to a generic dye. Then, using the well-established chemistry of dyeing fibers, the nanostructure is chemically bound to the fiber. The platform nature of the approach is illustrated by producing examples similar to those that have only been produced using customized synthesis techniques. Examples show the method can be utilized to attach gold nanoparticles, quantum dots, or grow porous MOF crystals on either natural or synthetic fibers.

Specifically, a reactive dye conjugating moiety, cyanuric chloride, was selected as a method to anchor nanostructures to the fiber substrate, as shown in FIG. 1b. In this process, the cyanuric chloride undergoes nucleophilic aromatic substitution with cellulose, and the remaining chlorine atoms are available for functionalization and provide a route to anchor additional nanostructures. This general approach is applicable to both nylon and cellulose. To illustrate this concept, a nylon swatch approximately 0.5″ (1.27 cm) square was functionalized with cyanuric chloride, reacted with cysteamine, and then exposed to 40 nm gold nanoparticles to bind the gold to the sulfur group anchored on the fabric surface.

The results of this process are shown in FIG. 2 with field emission scanning electron microscope (FESEM) images showing gold coating the fibers and in some areas gold completely covers the individual fiber strands. FIG. 2a shows gold coating the fabric surface such that in some cases individual fibers comprising the weave are no longer distinguishable due to a surface coating of gold. Magnification of an individual fiber, shown in FIG. 2c, shows gold covering the majority of a fiber strand surface. Also shown in FIG. 2, is the control group containing unmodified nylon, which highlights the changes in fiber texture with gold loading.

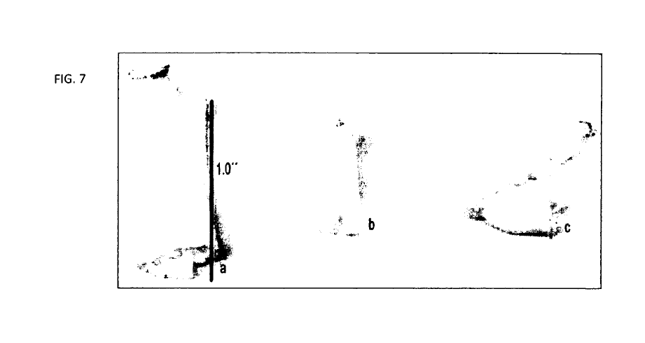

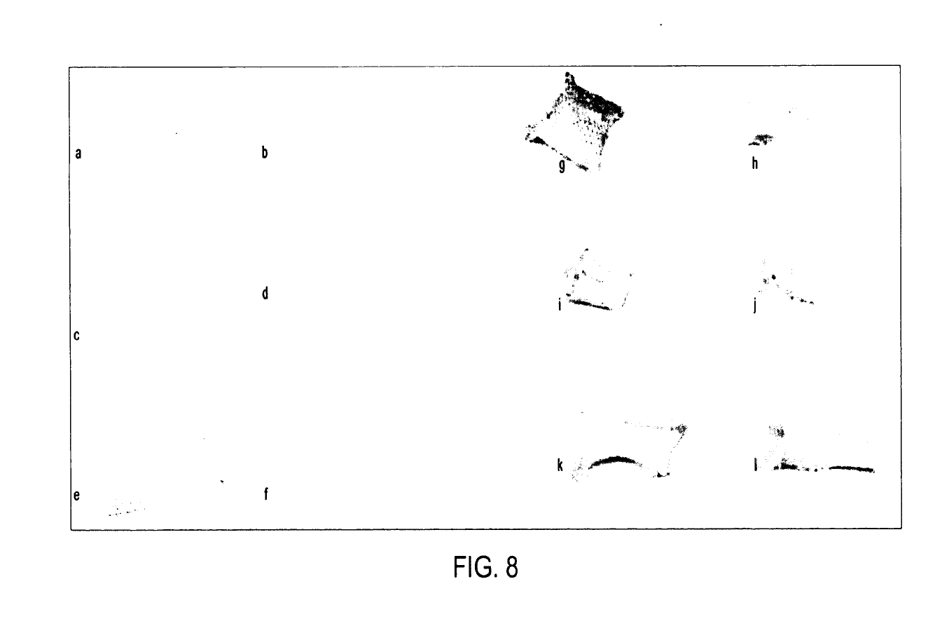

It is important to note that prior to taking the FESEM images, the fabric samples were rigorously washed first with chloroform, water, then commercial hand soap and water, and lastly acetone. The presence of the gold after rigorous washing highlights the robust chemical attachment of the metal to the surface of the particles. The ability of the gold nanoparticles to remain bound to the fiber substrate even with vigorous washing was in stark contrast with the fabric samples that were incubated with gold without the use of reactive dye chemistry, as shown in FIGS. 7-8.

To ensure that the coating observed on the fabric was gold, scanning electron microscopy energy dispersive x-ray spectroscopy (SEM-EDS) data were gathered and confirmed the presence of not only gold, but also sulfur, and chlorine. The presence of these elements is consistent with the reaction chemistry reflecting a thiol of cysteamine and residual chlorine of cyanuric chloride. SEM-EDS data on the gold modified samples were taken without gold sputtering, and additional SEM-EDS data are presented in the figures.

A distinct pink color, consistent with properties of gold, was observed when the nylon was functionalized with 40 nm gold as shown in FIG. 3a,b(41). The pink color was maintained on the fabric after washing the swatch with hot tetrahydrofuran (THF), water, and soapy water, which is consistent with the FESEM data showing robust chemical attachment of the gold to the surface of the fibers even after washing. Shown in FIGS. 3c and 3d are darker color changes resulting from the functionalization of the fabric with smaller 5 nm gold particles. Shown in FIG. 3e is the control nylon fabric that has been exposed to the same gold nanoparticle solution, but was not modified with the chemical attachment technology. The control showed no color change and was not stained by exposure to the gold nanoparticles. Specifically, the fabric in FIG. 3e was washed with hot THF and water, exposed to 5 nm gold overnight, and then the fabric was washed with hot THF and water. Nylon samples were also modified with 20 nm particles and provided a similar result as the 40 and 5 nm samples.

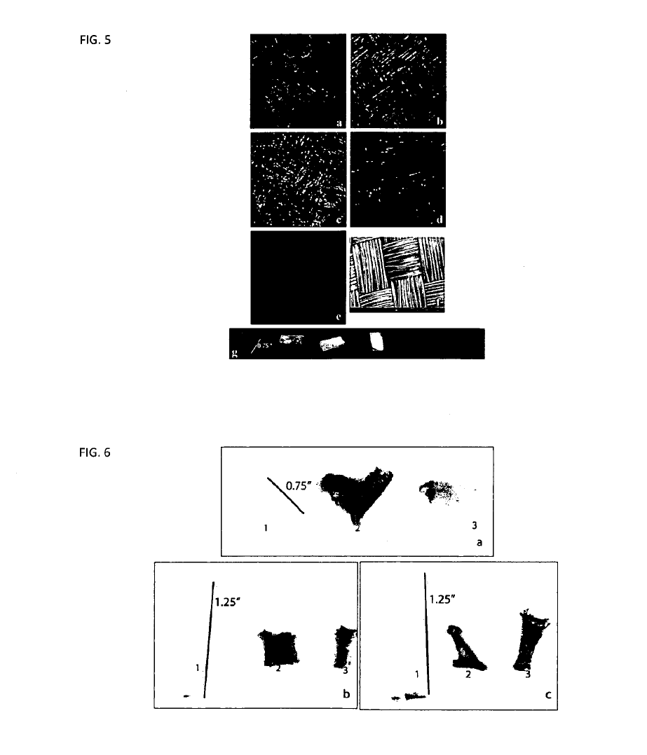

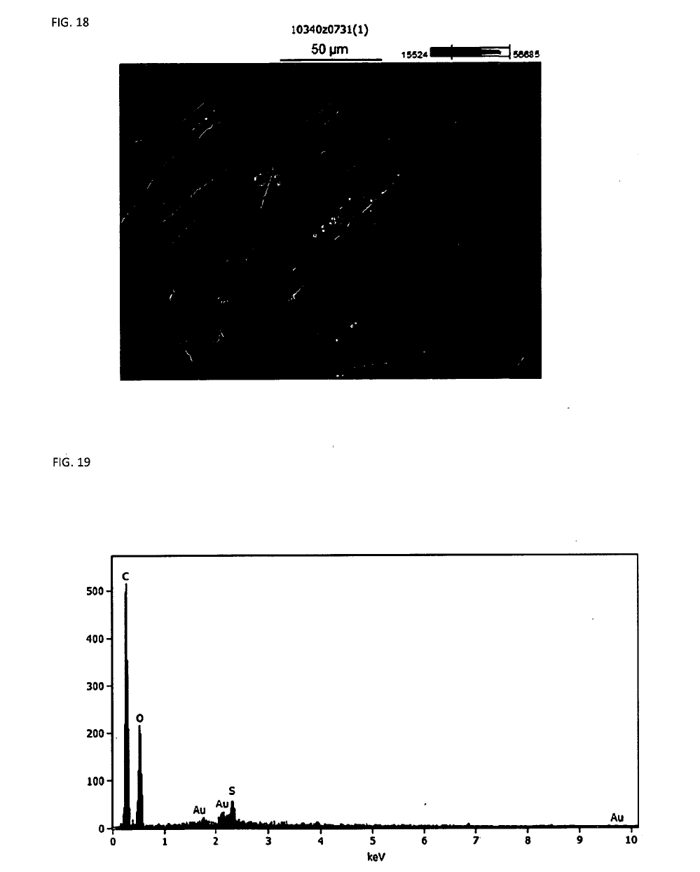

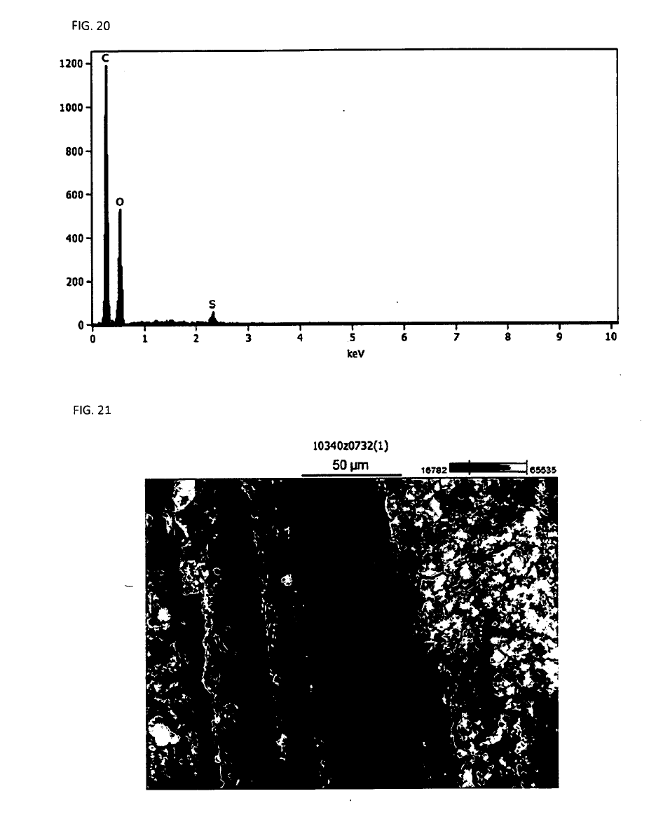

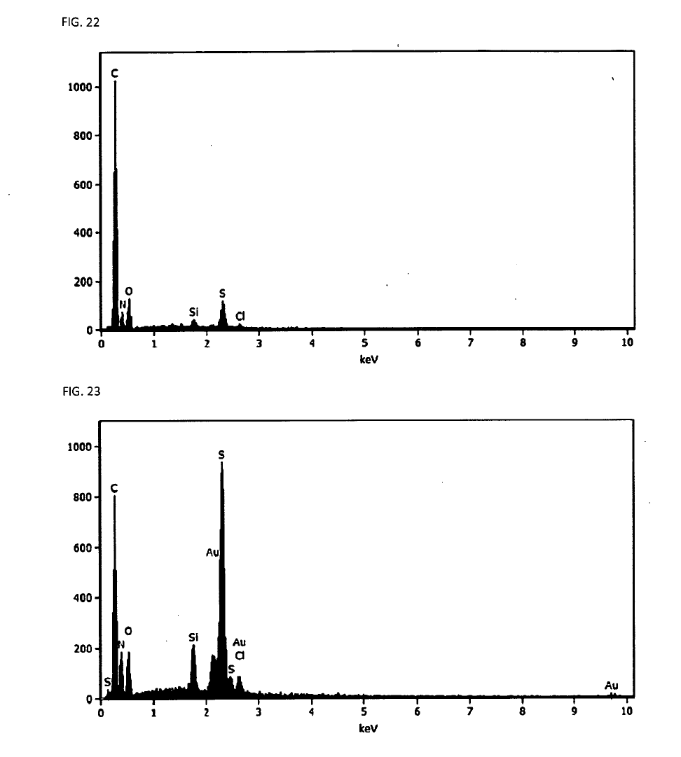

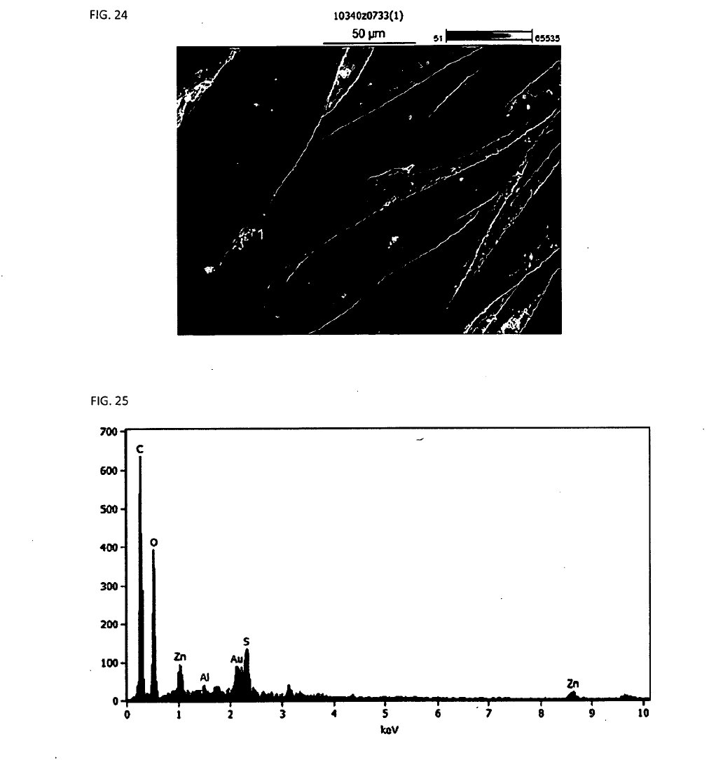

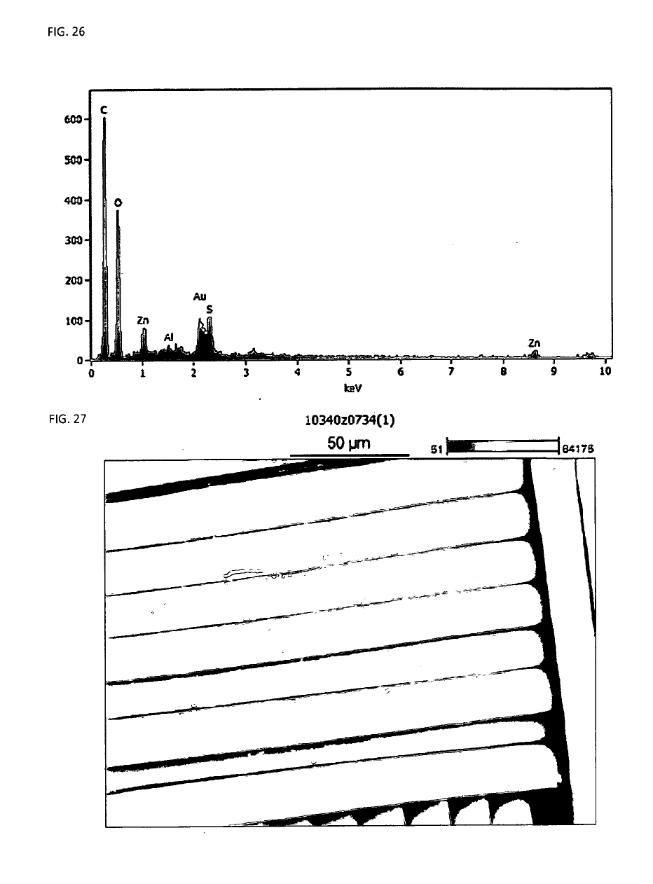

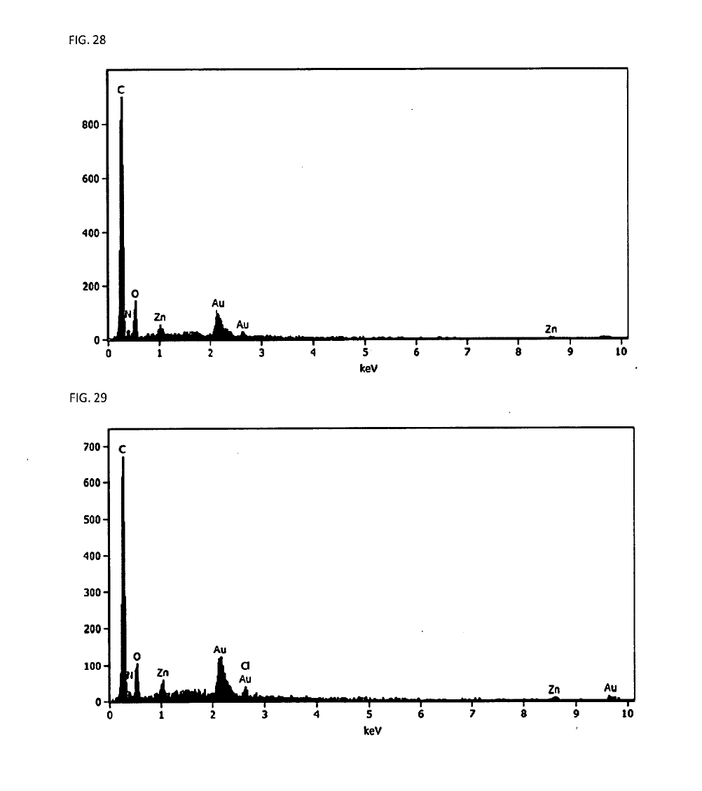

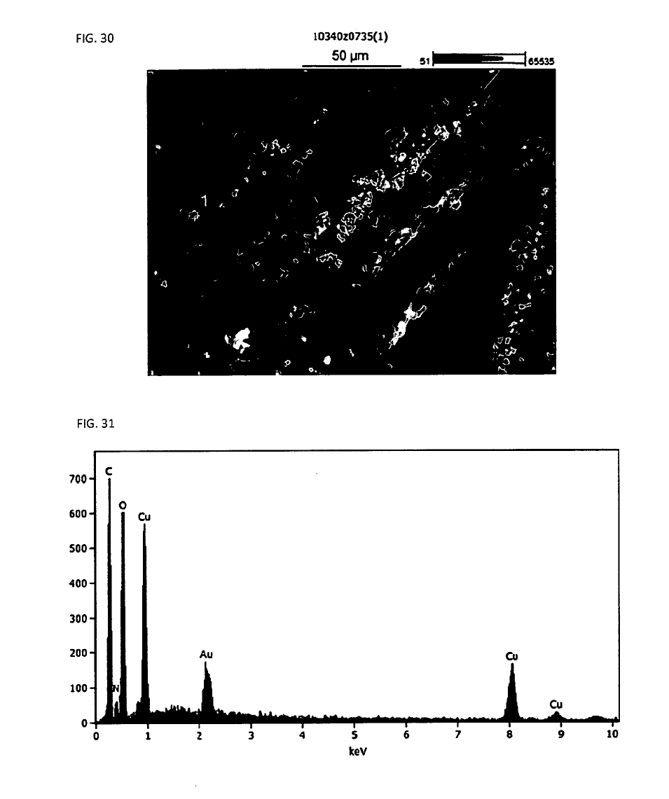

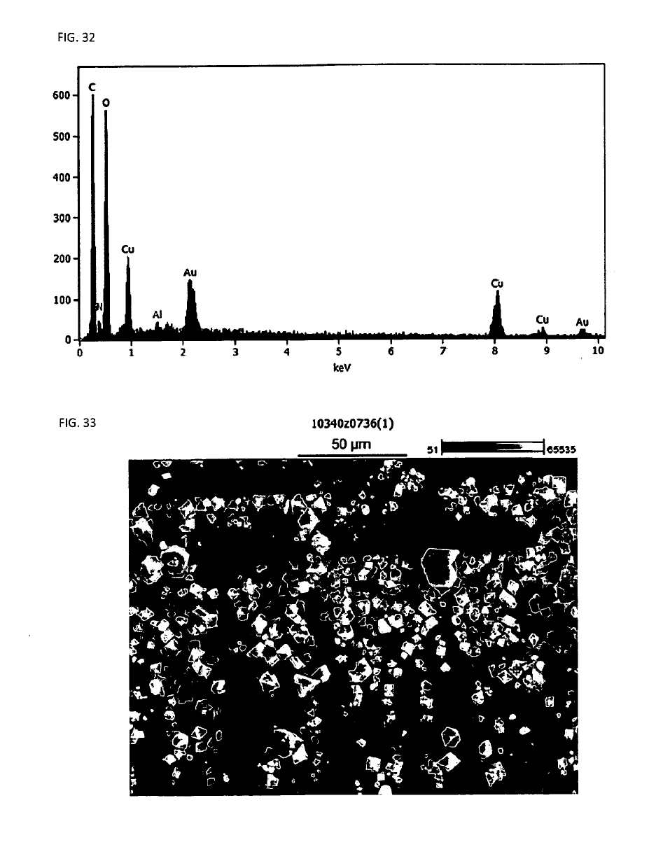

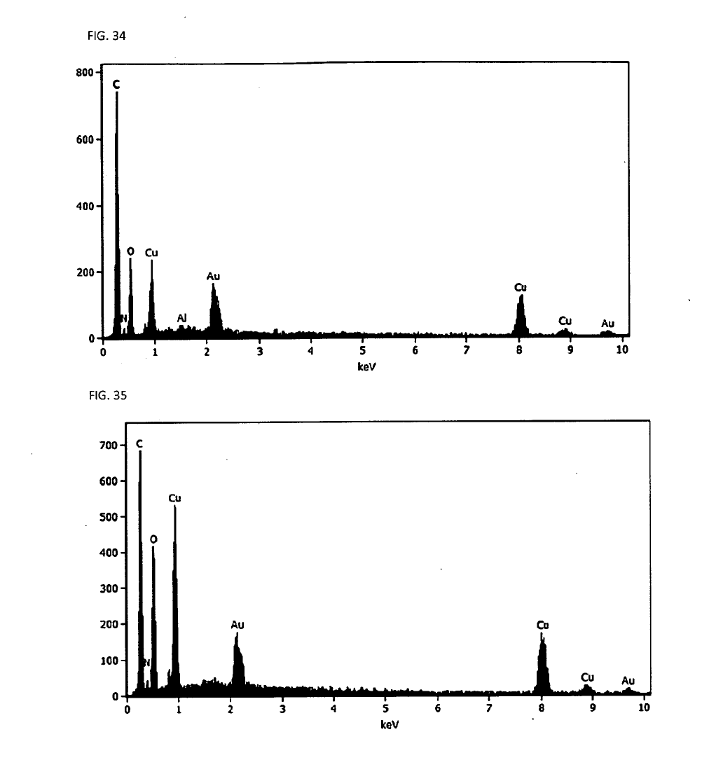

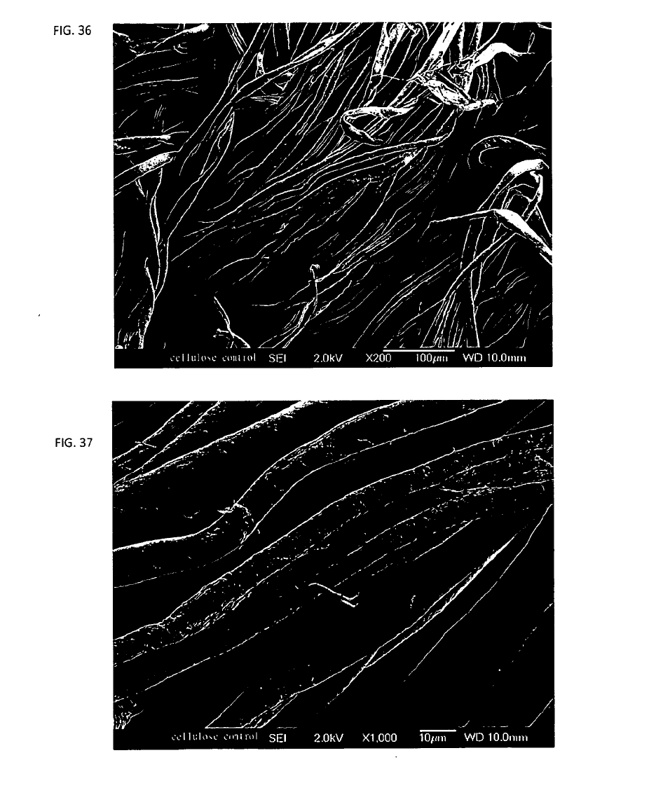

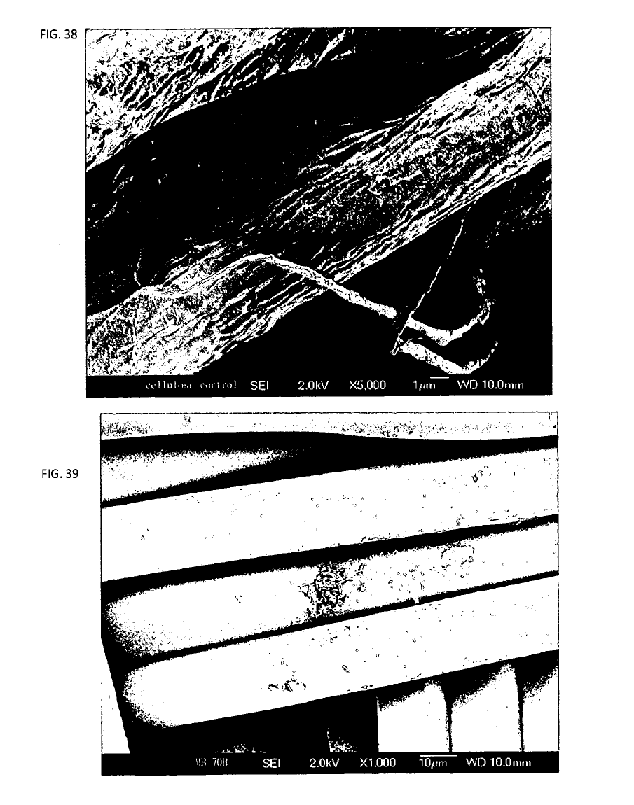

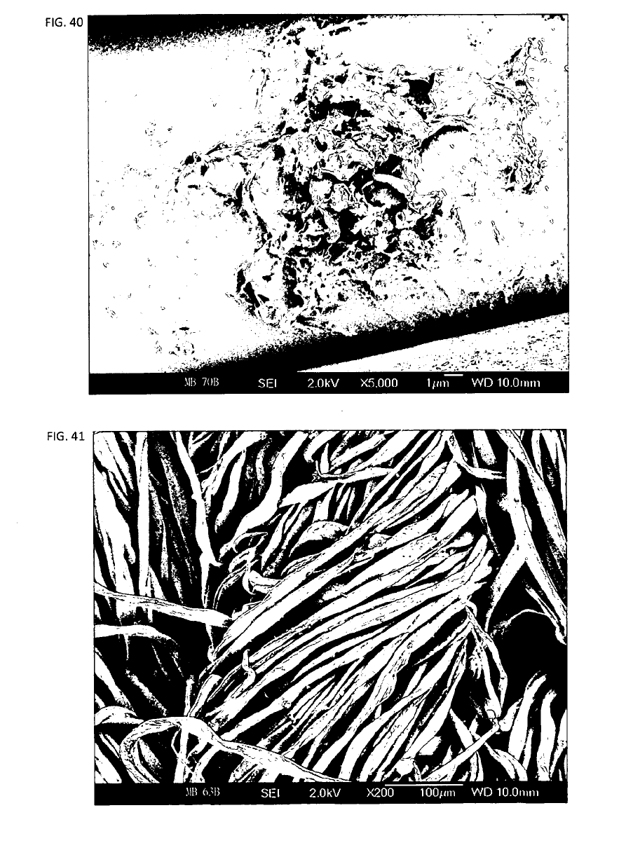

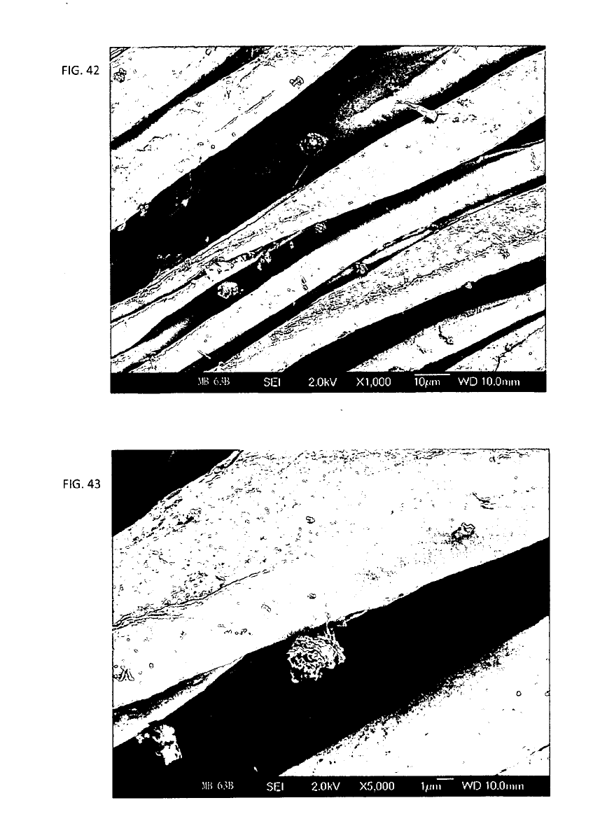

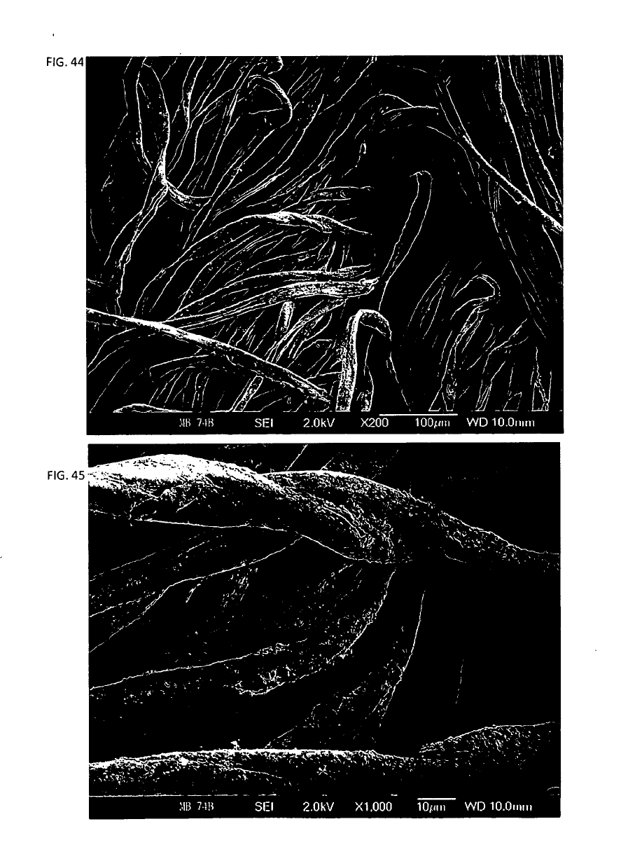

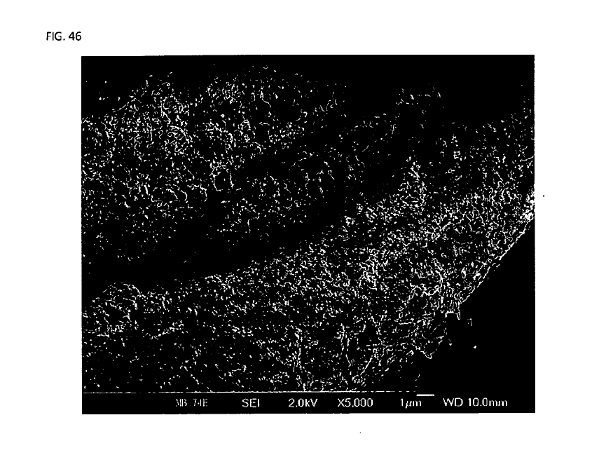

The reactive dye method (RDM) was also used to modify cellulose (cotton) swatches with 20 and 40 nm gold particles and produced similar results as the nylon samples. Images of cotton modified with gold are provided in FIGS. 6-9. The FESEM images of the cellulose modified with gold show significantly more surface texture on the modified fibers when compared to the original cellulose fibers. EDS data also verify the presence of gold on the surface of the cotton fibers. As with the nylon samples, cotton samples were washed with water, chloroform, soap and water, and acetone. Images and FESEM data of cellulose modified with gold are contained in FIGS. 18-20 and 44-46.

To quantify the total gold loading, microwave plasma atomic emission spectroscopy (MP-AES) measurements were completed on representative 40 nm gold-modified cotton and gold-modified nylon samples that were prepared. The gold loadings of these samples was nominally 0.077 wt. % for gold-modified cotton and 0.081 wt. % for gold-modified nylon.

Two similar processes were developed to bind gold to the surface of the fabric; one using cysteamine hydrochloride and the second using cysteamine. This modification is noted because the two processes produced similar results and the cysteamine hydrochloride salt is available at a significantly reduced price relative to the pure cysteamine reagent. The synthesis section of the work identifies these differences and highlights when each method was used. In this example the cysteamine has taken the place of the traditional chromophores and is available for additional reaction; however, other thiols, such as 6-amino-1-hexanethiol, could have also been used.

It is important to highlight that the fabric swatch was cut from a sample of nylon purchased from Jo-Ann's Fabric and Craft store, which illustrates that it is not necessary to have laboratory grade nylon and that this approach can be applied to currently industrially-produced fabrics. The RDM was also used to modify 0.5″ (1.27 cm) cellulose swatches. As was done for the nylon sample, a commercially available cellulose sample was obtained, in this case a T-shirt purchased from Wal-Mart (Faded Glory brand) reflecting the ease of application of this approach to existing commercially available fibers. Also, reactive dye chemistry is known to be applicable to wool and silk and in this work cellulose was selected as a representative example of these natural fibers. Likewise, illustration of the RDM to cellulose is broadly applicable to fabrics as well as other cellulose materials, such as wood or paper.

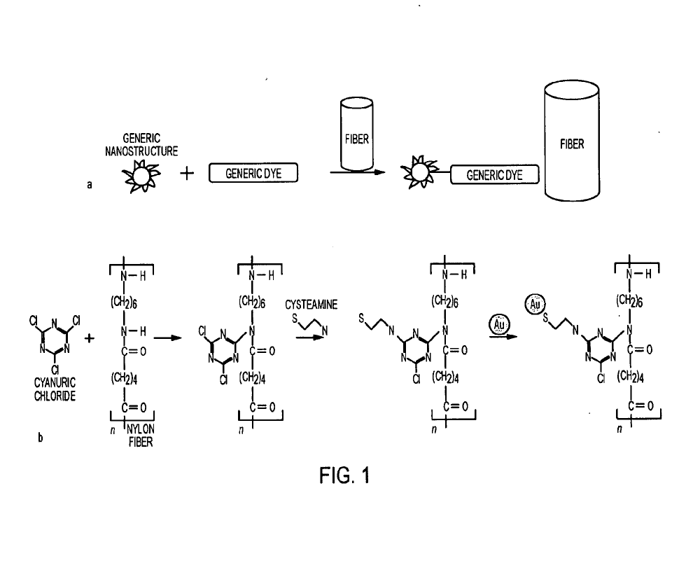

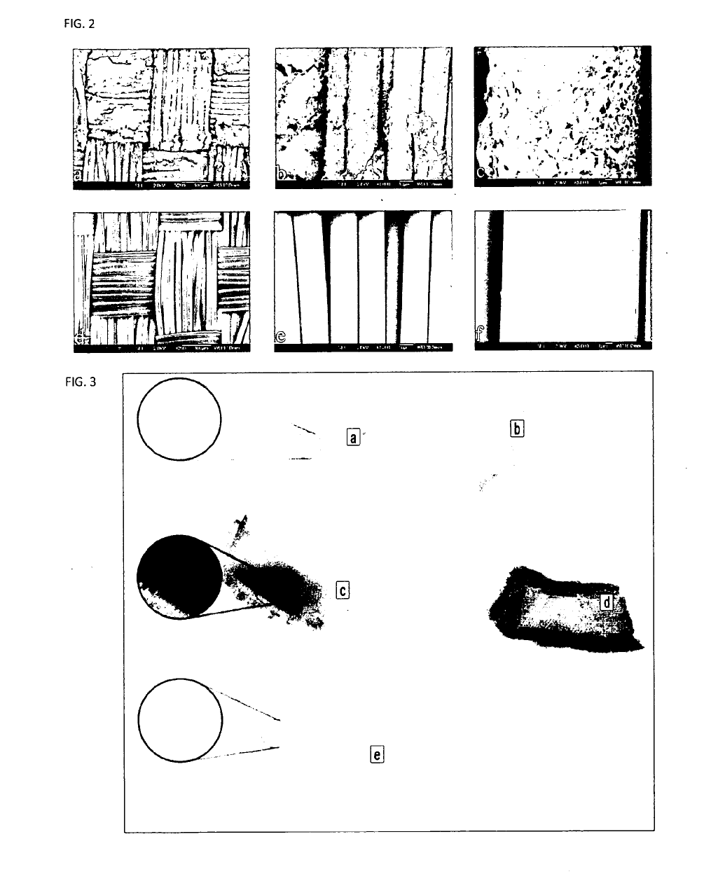

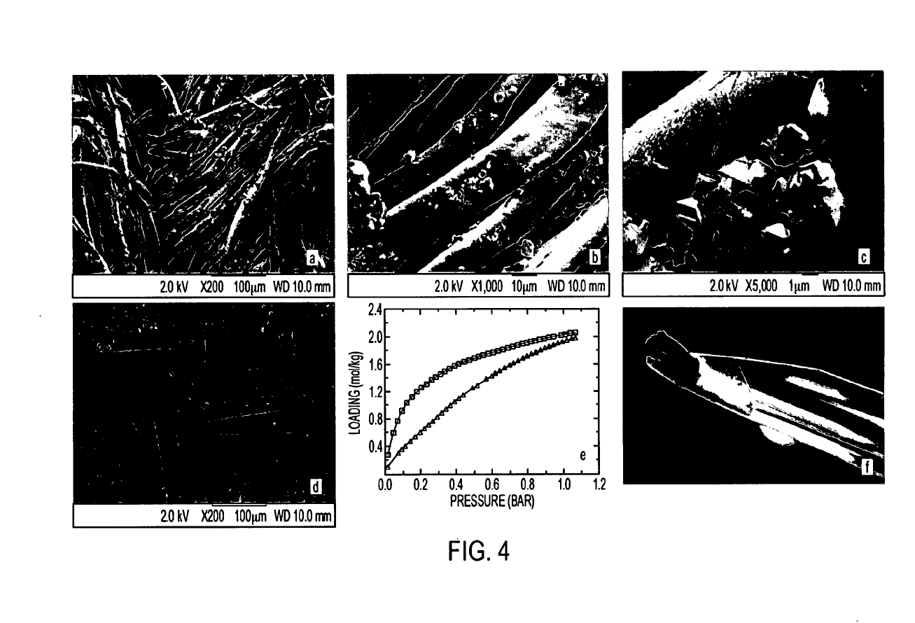

With the FESEM illustrating gold covering a large portion of the fiber surface (FIGS. 2, 18, 21, 44, 45, 46), it was hypothesized that these materials could provide a starting point for the construction of self-assembled monolayers or more complex nanostructures. To prove this concept, cellulose and nylon were modified to contain copper for the development of an organized crystalline copper-based metal organic framework (MOF). Using the same platform approach that was used to attach gold to fibers, a fabric sample was modified with cyanuric chloride, functionalized with cysteamine, and then copper was bound to the thiol. In this case the copper was added via copper nitrate to illustrate that the RDM approach does not specifically require a controlled metal nanoparticle in suspension. After the copper was added to the fabric using the RDM platform, the fabric was used as a metal structural building unit for the construction of a Cu-BTC MOF crystal as shown in FIG. 4.

The materials show high surface area with approximately 976 and 680 m²/g for cotton and nylon respectively, and X-ray diffraction (XRD) patterns consistent with Cu-BTC. As a control, Cu-BTC was prepared without the presence of fabric and resulted in a sample with 1778 m²/g. Compared to the pure Cu-BTC powder, the cotton and nylon have 55 and 38% of the pure powder surface area, respectively. Additionally, the presence of the fabric swatch functionalized to bind copper on the fabric did not impact the formation of MOF in the reaction solution. Specifically, Cu-BTC powder that formed in the reaction solution but was not attached to the fabric produced a sample containing 1760 m²/g of surface area.

To illustrate the viability of the bound nanostructure to perform industrially relevant separations, ethane and ethylene single component gas adsorption isotherms were measured and are shown in FIG. 4e. This example was selected because the separation of ethylene and ethane has been completed using membranes previously, which illustrates the potential application of the RDM to membrane fiber development (42). With copper as a Lewis acid, the fabric shows selectivity for the ethylene based on the available u electrons in the ethylene double bond (43). Selectivity for ethylene over ethane was observed for both nylon and cellulose modified Cu-BTC materials. Shown in FIG. 4f is a cellulose Cu-BTC fabric sample removed from the solution vial prior to washing with solvents. The Cu-BTC fabric was not washed with water due to Cu-BTC water instability(44). The color is consistent with the color of solvent filled Cu-BTC(45).

Also, the ability of the material to remove ammonia from air was evaluated by passing a humid air stream containing ammonia across the fabric and monitoring the effluent gas concentration in a gas breakthrough experiment (46). The data shows that the material readily captures ammonia from a flowing stream of humid air. Specifically, the cotton Cu-BTC sample adsorbed 2 mol/kg of ammonia in a 25° C. (50% relative humidity) air stream, and the nylon Cu-BTC sample produced a lower 0.5 mol/kg loading, which is consistent with the lower surface area of the Cu-BTC nylon sample.

To illustrate the application of RDM technology to optical nanostructures and surfaces, 6 nm CdSeS/ZnS alloyed quantum dots with a fluorescence of 505 nm were added to cellulose and nylon. FESEM images of these samples were completed; however, given the small size of the quantum dots it was difficult to see the particles using FESEM, as shown in FIG. 5f. Some large particle aggregations were observed on the fibers in some images and EDX data indicated Zn in these aggregations, which is consistent with the CdSeS/ZnS composition of the quantum dot.

To observe the loading of the quantum dots on the fabric, maximum intensity confocal microscopy images were gathered as shown in FIG. 5. By measuring controls of the native quantum dots in solution, the fabric, and the composite, the images were colored to show unmodified nylon fiber and quantum dot florescence in dark blue and yellow respectively. As with the gold sample, the coverage on the fibers is high. For these samples, FIG. 5a shows the loadings after washing with n-hexane, water, and chloroform, but without washing with soap. FIG. 5b shows the loading of the particles after washing with n-hexane, water, chloroform and with soap, water, and acetone. The loadings of the quantum dots in FIG. 5a are consistent with the loadings of gold observed via FESEM on nylon with large deposits of quantum dots completely covering the fibers in the nylon weave. The sample in FIG. 5a was prepared using cysteamine (process 3a). When this sample was washed with soap, however, the large deposits appear more distributed across the fibers but the quantum dots remain well attached to the fabric. There was no visual difference in these two samples, 5a or 5b, with both maintaining a strong green color consistent with the quantum dot stock solution.

A second sample was prepared using cysteamine hydrochloride (process 3b) and the results are shown in FIGS. 5c and d, with FIG. 5c showing the sample after washes with n-hexane, water, chloroform, and 5d after an additional washing with soap and water and acetone. Prior to washing with soap the quantum dots cover the nylon fibers very evenly. After washing with soap, some loses are observed; however, quantum dots are still present. Visually, the sample shown in FIGS. 5c and d were both green, consistent with the stock quantum dot solution. The sample washed with soap (5d), however, was a slightly lighter green in color.

Although these two samples showed variance in how the quantum dots load the nylon fibers, both produced nylon swatches with bound quantum dots and florescence consistent with the stock quantum dot solution.

The optical measurements also confirmed that the observed fluorescence of the quantum dots in solution has not changed upon binding to the fiber surface. Specifically, a sample of the solution that was used as the source of the quantum dots for the experiments shown in FIG. 5, was placed on a glass slide and imaged using the same settings used to image the fibers. The quantum dot solution fluoresced at 505 nm using a 405 nm laser for excitation and the observed fluorescence of the quantum dots bound to the fabric was also 505 nm showing, as expected, that the RDM of attachment has not altered the fluorescence.

The RDM was repeated using quantum dots with fluorescence wavelengths of 525, 575, 630, and 665 nm producing fabrics of different colors as shown FIG. 5e. The fibers in FIG. 5e were washed with n-hexane, water, chloroform, soap and water, and acetone. The presence of fluorescence after these washings emphasizes the robust attachment of the nanoparticles to the fibers.

To demonstrate the viability of attaching a metal ion to the fiber using the reactive dye chemistry, and subsequently reducing the ion to a ZVM, cotton was coated with gold ions. A roll of cotton fabric was exposed to gold ions after pretreatment with the reactive dye method. Another cotton fabric sample was pretreated with the reactive dye method, exposed to gold ions, washed with water, and then reduced to form zero valent gold. A slight pink color was present in the reduced sample, indicating zero valent gold on the fabric.

The results discussed illustrate the use of reactive dye chemistry to attach nanostructures to fibers. The approach is applicable to synthetic and natural fibers and can be used as a starting point for the assembly of complex nanostructures, such as MOFs on fibers. Likewise, the gold in the modified fibers is available for surface chemistry reactions and provides a starting point for applying other gold-based nanotechnology, such as self-assembled monolayers, to fibers. The MOF-modified fibers show selectivity for ethylene over ethane and can remove ammonia from a humid air stream. The quantum dot fibers fluoresce with the same wavelength of the bulk solution even though the particles are bound to the surface. The gold and quantum dot examples provided survive not only solvent washes, such as acetone, chloroform, and hexane, but also soap and water. The MOF modified fibers survive washes with solvents, but were not washed with water due to Cu-BTC instability. These results provide three examples of utilizing reactive dyes to modify synthetic and natural fibers, and provide a route to move nanotechnology from a lab practice to commercially available substrates, such as fabrics, papers, and plastics.

### Materials and Methods:

### Materials Synthesis

Although other dyes can be used, in the cases shown, cyanuric chloride was covalently attached to the fiber substrate. Then the attached cyanuric chloride was modified with an amino thiol bonding agent, and lastly a functional nanostructure such as a metal, metal salt, or quantum dot, was added to the modified fiber. The order of these steps can vary and may impact the quality and properties of the product produced.

Two variants of this method were used to prepare most of the fabrics. The processes are similar with the first process using cysteamine hydrochloride as the bonding agent and the second process using cysteamine as the bonding agent. Two methods were examined because cysteamine hydrochloride is less expensive than pure cysteamine. Both methods produced similar results. The use of cysteamine hydrochloride was illustrated by modifying nylon with gold as detailed in process 1b, nylon modified with MOF as described in 2b, and nylon modified with quantum dots as detailed in process 3b. The gold was purchased as nanoparticles in solution from Strem Chemicals Inc. and used as received.

In all cases these materials were prepared separately (nylon and cellulose were not placed together in the reaction vials). Cellulose was prewashed with soap and water and then rinsed with chloroform. The nylon was not prewashed but the other processing steps are the same for both cellulose and nylon.

The following protocol was followed to observe the persistence of the nanostructure on the fiber substrate after washing. Washing of the nanostructure-modified fabric was completed with enough solvent to completely submerge the fabric in solvent. The solvent and fabric swatch were mixed using a scapula. If color was observed in the solvent upon washing, the fabric was subsequently rinsed with more solvent using a wash bottle and washed again. Solvents, water, and soap were washed at room temperature. When hot THF was used the temperature was 40-50° C. Washing of the nanostructure modified fabric was completed with enough solvent to completely submerge the fabric in solvent. The solvent and fabric swatch were mixed using a microspatula. If color was observed in the solvent upon washing, the fabric was subsequently rinsed with more solvent using a wash bottle and washed again. To complete a wash using soap the fabric swatch was placed in the palm of the hand and hand soap applied; the fabric was then rubbed against the palm of the hand with one's index finger. Different solvent washes were used for different attached structures (optimized to try to remove the attached structure). For gold the washings were completed using hot THF (40-50° C.), water, soap (hand soap), and acetone. In some cases for gold the washings were water, chloroform, soap, water, and acetone. STOKO hand soap was used to wash the fabrics. STOKO hand soap contains: water, sodium laureth sulfate, cocamidopropyl betaine, PEG-200, hydrogenated glyceryl palmate, PEG-7, Glyceryl Cocoate, Undecylenamidopropyltrimonium, methosulfate, fragrance, benzyl alcohol, methylchloroisothiazolinone, methylisothiazolinone, citric acid, and blue 1.

Per the manufacture, the gold nanoparticles solutions are supplied at an optical density of 1 in a solution stabilized with citrate buffer (0.1 mg/ml). The concentration of the gold particles at these conditions are 6.94×10⁻², 5.31×10⁻², and 4.65×10⁻² mg/ml for 5, 20, and 40 nm particle solutions, respectively.

Process 1a: Modification of Nylon or Cellulose with Gold Via Cysteamine

The procedure below applies to both cellulose and nylon. This process was used to produce the sample shown in FIG. 2a-c as well as used to produce samples for MP-AES analysis. To begin, 2 swatches of nylon or cellulose, approximately 0.5″ (1.27 cm) square, were cut from a yard of nylon or from the purchased cotton T-shirt, respectively. The swatches weighed nominally 0.015 g and 0.0205 g for nylon and cellulose, respectively. The fabric was then placed in a 100 mL beaker containing 5 g of sodium carbonate dissolved in 50 mL of water at 65° C. and stirred for 5-10 min. In an Erlenmeyer flask, 1.88 g of cyanuric chloride was dissolved in 40 mL chloroform and stirred using a stir bar. The fabric was added to the flask, and a rubber stopper with a syringe needle for ventilation was placed on the flask. The fabric was stirred for 1 hour, and in a separate Erlenmeyer flask, 20 mL of water and 0.43 g of cysteamine were added. After the fabric was stirred in the cyanuric chloride solution for 1 hour, the fabric was taken out and added to the flask containing cysteamine, a ventilated stopper was placed on the flask, and the fabric was stirred for 22 hours. The fabric was removed from the cysteamine solution and placed in a 30 mL beaker to be washed. The fabric was washed with water and chloroform. Gold nanoparticles were then added to the fabric in excess such that the gold solution submerged the fabric. The beaker was covered with paraffin film and sat overnight. The fabric was then washed with water and chloroform to remove gold particles not covalently bound to the fabric. This process was repeated for each washing fluid. In most cases, to determine the impact of soap and water washing, the fabric was then cut in half and one half was placed in a 20 mL vial that was partially covered with paraffin film to allow it to dry. The other half was washed with soap and water and rinsed with acetone and then placed in a 20 mL vial that was partially covered with paraffin film to dry.

Process 1b: Modification of Nylon with Gold Via Cysteamine Hydrochloride

This process was used to produce the sample shown in FIG. 3. To begin, swatches of nylon or cellulose, approximately 0.5″ (1.27 cm) square, were cut from a yard of nylon or from the purchased cotton T-shirt.

In an Erlenmeyer flask, 40 mL of tetrahydrofuran (THF) was heated (approximately 40-50° C.) and 1.13 g cysteamine hydrochloride added. Once the cysteamine hydrochloride dissolved, the heat was removed and 0.92 g of cyanuric chloride was added. In a beaker, a solution of 0.4 g of sodium carbonate in 4 mL of water was prepared, added to the THF mixture, and allowed to sit overnight. The next day two swatches of the nylon were pretreated in a solution of 5 g sodium carbonate and 50 mL of water at 60° C. for 5-10 minutes. After pretreatment, the fabric swatches were added to the THF mixture and allowed to sit for 3 days. Next, one of the three pieces was washed with hot THF and water. A gold (5 nm) solution was added to the fabric in a beaker and the solution was allowed to sit overnight covered. Gold nanoparticles were added to the fabric in excess such that the gold solution submerged the fabric. The next day the fabric was removed from the gold solution and washed with hot THF and water. A second swatch of fabric that was stirred for 3 days was collected from the reaction solution and washed with hot THF, water, soap and acetone; then 5 nm gold solution was added to the fabric, and the fabric/gold mixture was allowed to sit covered overnight. Lastly the second fabric swatch was then washed with hot THF, water, soap, and acetone. The results of this process are shown in FIGS. 3c and 3d.

The same reaction process was repeated to produce the results shown in FIGS. 3a and b. In these experiments, however, the reaction times to attach the reactive dye conjugating moiety to the fiber substrate and the time to bind the gold to the reactive dye conjugating moiety, were reduced to 1 hour instead of overnight or multiday times. Specifically, following the process above, cysteamine hydrochloride was dissolved in THF and cyanuric chloride was added. A solution of water and sodium carbonate was added to the flask and allowed to stir for 1 hour. Nylon was then pretreated in a solution of an aqueous solution of sodium carbonate (5 g in 50 mL water) and then added to the reaction mixture. After stirring for one hour, the nylon was taken out and washed with hot THF and water. Next, a 40 nm gold solution was added to the fabric and the fabric solution was allowed to sit covered for one hour. It was then collected and washed with hot THF and water. The fabric was cut in half, one half was saved in a 20 mL vial that was partially covered to dry and the other half was washed with soap and water, then with acetone, and then added to a vial to dry.

Process 2a: Modification of Nylon and Cellulose with a MOF Via Cysteamine

This process was used to produce the cellulose modified samples shown in FIGS. 4a-c and 4e and f. This process is also applicable to nylon. To begin, 4 swatches of nylon or cellulose, approximately 0.5″ (1.27 cm) square, were cut from a yard of nylon and from the purchased T-shirt respectively. One piece of nylon weighed approximately 0.0148 grams and one piece of cellulose weighed approximately 0.0205 g.

The nylon and cellulose fabric was pretreated by stirring the fabric in a solution of 50 mL of water and 5 g of sodium carbonate for 5-10 minutes at approximately 60° C. Separately, 40 mL of chloroform and 1.84 g of cyanuric chloride were added to an Erlenmeyer flask; the fabric was then removed from the pretreatment solution and placed in the Erlenmeyer flask. The flask was capped with a rubber stopper and needle for ventilation and allowed to stir for one hour. Then in another flask 20 mL of water and 0.43 g cysteamine were mixed. After mixing in the cyanuric chloride solution for an hour, the fabric was removed from the flask and placed in the cysteamine solution and allowed to stir for 22 hours. Afterwards, the fabric was removed and washed with chloroform and water. In a separate 20 mL vial, 0.8 g of copper nitrate trihydrate was added to 6.6 mL of water and stirred until copper (II) nitrate was dissolved. Then the fabric was added to the copper (II) nitrate solution and the mixture was allowed to sit for approximately 24 hours.

A Cu-BTC solution of 1,3,5-benzenetricarboxylic acid (0.4 g), copper nitrate (0.8 g), and 6.6 mL each of water, ethanol, and N,N-dimethylformamide was prepared and allowed to stir for 15 minutes. The Cu-BTC solution was separated into two 20 mL vials, each containing approximately half the solution volume. A fabric swatch was removed from the copper nitrate solution and placed in one of the vials containing the Cu-BTC reaction solution. A second swatch was removed from the copper nitrate solution and placed in the other Cu-BTC reaction solution vial. The Cu-BTC vials were tightly sealed and baked in the oven for 20 hours at 85° C. The vials were removed from the oven, the contents of the two vials were combined into one vial, and then allowed to sit to settle the MOF powder from the solution, and lastly the solvent was removed leaving the crystals and fabric. Dichloromethane was then added to the vial and allowed to sit overnight (solvent exchange). The solvent exchange process was repeated 3 times, each time removing the majority of the dichloromethane solvent and replacing it with fresh dichloromethane.

Process 2b: Modification of Nylon with an MOF Via Cysteamine Hydrochloride

A 0.5″ (1.27 cm) size swatch of nylon (approximately 0.0148 g) was cut from a yard of nylon. Next, 40 mL of chloroform and 1.84 g of cyanuric chloride were placed in an Erlenmeyer flask. Then an aqueous Na₂CO₃ solution was prepared (0.8 g sodium carbonate in 8 mL water). In a second beaker, an aqueous cysteamine solution was prepared (1.2 g cysteamine hydrochloride in 5 mL water). The Na₂CO₃ solution was added to the cysteamine hydrochloride solution. This mixture was then added dropwise to the stirring solution of cyanuric chloride over 10 min and the solution was stirred overnight. The next day the nylon was pretreated by stirring the fabric in a solution of 50 mL of water and 5 g of sodium carbonate for 5-10 minutes at approximately 60° C. The pretreated nylon was then added to the cyanuric chloride mixture and allowed to stir in the reaction mixture for 4 days. The fabric was then taken out of the mixture, washed with water and chloroform. In a separate 20 mL vial, 0.8 g of copper (II) nitrate trihydrate was added to 6.6 mL of water and stirred until copper nitrate was dissolved. Then the fabric was added and allowed to sit for approximately 24 hours.

A Cu-BTC solution of 0.4 g 1,3,5-benzenetricarboxylic acid, 0.8 g copper nitrate, and 6.6 mL each of water, ethanol, and N,N-dimethylformamide was prepared and stirred for 15 minutes. The Cu-BTC solution was separated into two 20 mL vials each containing approximately half the solution volume. A fabric swatch was taken out of the water and copper nitrate solution and was placed in one of the vials containing the Cu-BTC reaction solution. A second swatch was removed from the water copper nitrate solution and placed in the other Cu-BTC reaction solution vial. The Cu-BTC vials were tightly sealed and baked in the oven for 20 hours at 85° C. The vials were removed from the oven and combined into one vial. After the crystals had settled to the bottom of the vial, the solvent was pipetted off of the crystals and fabric. Dichloromethane was then added to the vials and allowed to sit overnight (solvent exchange). The solvent exchange process was repeated 3 times with each time removing the majority of the dichloromethane solvent and replacing it with fresh dichloromethane. This process was used to produce the samples shown in FIG. 3d.

Process 3a: Modification of Nylon and Cellulose with Quantum Dots Via Cysteamine

This process was used to produce the sample shown in FIGS. 5a and b. To begin, 3 swatches of nylon, approximately 0.25″ (6.35 mm) square, were cut from a yard of nylon with 1 piece weighing nominally 0.005 g. When this process was applied to cellulose, 3 pieces approximately 0.25″ (6.35 mm) square were cut from the purchased T-shirt with one piece weighing approximately 0.011 g.

Cellulose and nylon were pretreated in 50 mL water and 5 g of sodium carbonate at 65° C. for 5-10 minutes. The fabric was then added to an Erlenmeyer flask containing 40 mL of chloroform and 1.84 g of cyanuric chloride, and the flask was covered with a rubber stopper that contained a needle for ventilation and stirred for one hour. After one hour of stirring, the fabric was moved to a new flask that contained 0.45 g of cysteamine dissolved in 20 mL of water. The flask was covered with a rubber stopper that contained a needle for ventilation and allowed to stir for 22 hours. After 22 hrs, the fabric was taken out, put in a beaker, and washed with chloroform and water. Quantum dots were then added to the fabric in excess such that the quantum dot solution submerged the fabric. The vial containing the fabric and quantum dots was covered with paraffin film. The paraffin film contained a small hole to allow for evaporation, and the fabric sat at ambient conditions until all liquid had evaporated (approximately 5 days). The fabric was then washed with n-hexane, water, and chloroform. After washing, the fabric was cut in half, and one half was saved in a 20 mL vial and the other half was washed with soap, water and acetone and lastly placed in a 20 mL vial to dry.

Process 3b: Modification of Nylon with Quantum Dots Via Cysteamine Hydrochloride

This process was used to produce the sample shown in FIG. 5c, d, and f. To begin, 4 swatches of nylon, approximately 0.5″ (1.27 cm) square, were cut from a yard of nylon. A 0.5″ size swatch of nylon weighs approximately 0.0148 g. Next, 40 mL of chloroform and 1.84 g of cyanuric chloride were placed in an Erlenmeyer flask. In a beaker, 8 mL of water and 0.8 g sodium carbonate were added together and stirred until the sodium carbonate dissolved. In a second beaker, 5 mL of water and 1.2 g cysteamine hydrochloride was added and stirred until the cysteamine hydrochloride dissolved. The sodium carbonate mixture was added to the cysteamine hydrochloride mixture. This mixture was then added drop wise to the cyanuric chloride mixture over a 10 minute time span and then was stirred overnight. The next day the nylon was pretreated for 5-10 minutes with a solution of 5 g sodium carbonate in 50 mL of water at 60° C. The fabric was then removed from the pretreatment and placed in the reaction mixture and stirred for 4 days. The fabric was then taken out of the mixture, washed with water and chloroform, and a quantum dot solution was then added to the fabric such that the quantum dot solution submerged the fabric. The fabric solution was covered, and allowed to sit for two nights until the liquid was evaporated. The fabric was then washed with n-hexane, water, and chloroform. The washed fabric swatch was then cut in half and one half was saved in a vial. The other half was washed with soap and water and then acetone.

### Ingredients of Soap

Hand soap was used to wash the fabrics. The soap contains: water, sodium laureth sulfate, cocamidopropyl betaine, PEG-200, hydrogenated glyceryl palmate, PEG-7, Glyceryl Cocoate, Undecylenamidopropyltrimonium, methosulfate, fragrance, benzyl alcohol, methylchloroisothiazolinone, methylisothiazolinone, citric acid, and blue 1.

### Adsorption Measurements

Gas phase adsorption isotherms were completed using a Micromeritics ASAP 2020. The Cu-BTC fabric samples were outgassed at 170° C. temperature at 1.0° C./min ramp rate overnight under vacuum on a Schlenk line. Ethylene and ethane adsorption isotherms were completed at 298 K by using a closed loop recirculating water bath to maintain a constant 25° C. temperature on the adsorption sample cell.

### Confocal Microscopy Measurements and Image Analysis

Samples were imaged using an A1 laser-scanning confocal microscope equipped with a spectral detector (Nikon Instruments), using a 20× objective (Plan Fluor 20× MImm DIC N2, Nikon Instruments). Fluorescence was excited using a 405 nm laser, and spectral emission was detected from 410-720 nm, in 10 nm increments. A laser power of 0.9 (out of 100), a spectral detector high voltage (HV) of 150, and a confocal pinhole diameter of 3.0 airy disc units were used. A pixel size of 1.24 μm/pixel was used in the x, and y directions, with a scan size of 1024×1024 pixels, and a scan speed of 0.5 frames/second (pixel dwell time of 1.1 μs). For each fabric sample, three-dimensional image stacks (Z-stacks) were acquired using a step size of 2.5 μm. Samples of unlabeled nylon and cellulose fabrics, as well as quantum dots in solution, were acquired and used as single-label controls for linear unmixing (described below). Images were saved in lossless, nd2 file format.

Spectral image stacks were linearly unmixed using NIS Elements software (Nikon Instruments). Prior to unmixing, pure (end-member) spectra of each fluorescent species was measured from a field of view from each single-label control sample (cellulose, nylon, and quantum dots in solution). Linear unmixing was applied to each fabric-quantum dot sample using a spectral library containing either nylon and quantum dot spectra (for treated nylon fabrics) or cellulose and quantum dot spectra (for treated cellulose fabrics). Unmixed Z-stacks were then processed using a maximum intensity projection. Maximum intensity projections were saved in lossless format as both nd2 and tiff files.

### Microwave Plasma Atomic Emission Spectroscopy

An Agilent 4100 MP-AES microwave plasma atomic emission spectrometer was used for the elemental analysis. The instrument features a magnetically excited microwave plasma source operating on nitrogen gas, and an onboard argon bottle for plasma ignition. The nitrogen gas is provided by means of an Agilent 4107 nitrogen generator. An Agilent G8480A, SPS-3, 180 position auto-sampler was used to hold blanks, standards and samples. A blank (deionized water) and three gold standard samples, with a concentration of 10, 1, and 0.1 ppm, were used as calibration standards.

### Digestion of Cellulose

A gold dyed fabric swatch was cut up into small pieces, weighed, and then put in a beaker. Then 5 mL of deionized water and 5 mL of concentrated nitric acid were added. A glass dish was put over the beaker and it was put on a hot plate and allowed to boil for approximately 15 minutes. The beaker was then removed from the hot plate and an additional 5 mL of concentrated nitric acid was added. The beaker was returned to the hot plate and the solution boiled for approximately 15 minutes. Then 5 mL of concentrated nitric acid was added to the beaker, the beaker was placed back on the hot plate, and allowed to boil for approximately 15 minutes. The beaker was taken off the hot plate to cool. Upon cooling, 5 mL hydrogen peroxide (30%) was added slowly to the cellulose. The beaker was brought back to boil for 15 minutes. Then 5 mL of hydrochloric acid was added to the cellulose and boiled for 15 minutes. Both fabric solutions were filtered and added to a 100 mL volumetric flask. Deionized water was added until the solution reached the 100 mL line. The flask was shaken and then moved to a bottle to store. This process was used for both the plain fabric swatches and the fabric swatches with gold attached to them.

### Digestion of Nylon

A gold dyed fabric swatch was cut up into small pieces, weighed, and then put in a beaker. Then 5 mL of deionized water and 5 mL of concentrated nitric acid were added. A glass dish was put over the beaker and the mixture was boiled for approximately 15 minutes. The beaker was then removed from the hot plate and an additional 5 mL of concentrated nitric acid was added. The beaker was returned to the hot plate and the solution boiled for approximately 15 minutes. Then 5 mL of concentrated nitric acid was added to the beaker. Then 5 mL of hydrochloric acid was added to the nylon. Unlike the cellulose, it was not necessary to boil the solution another 15 minutes after the addition of the last portion of concentrated nitric acid or hydrochloric acid. Both fabric solutions were filtered and added to a 100 mL volumetric flask. Deionized water was added until the solution reached the 100 mL line. The flask was shaken and then moved to a bottle to store. This process was used for both the plain fabric swatches and the fabric swatches with gold attached to them.

### Field Emission Scanning Electron Microscopy

Samples were prepared by cutting sub-samples from each sample, mounting on double-stick carbon tape and gold sputter coating. Images of gold modified fabrics were analyzed uncoated by low vacuum SEM and SEM-EDS. SEM-EDS was performed using a JEOL JSM-6490LV scanning electron microscope coupled to a Thermo Scientific Noran System SIX energy dispersive x-ray spectrometer system operating in low vacuum mode.

### Powder X-Ray Diffraction

Powder X-ray diffraction (PXRD) data were recorded using a Rigaku MiniFlex600 with a Dtex detector. Samples were scanned at 40 kV and 15 mA using Cu Kα radiation and a scan step size of 2θ=0.02° over a range of 3-60° 2θ.

### Ammonia Microbreakthrough

Ammonia microbreakthrough experiments were run on fabric samples to determine the efficiency of deposition of the MOF onto the substrate. As a comparison, a packed bed of Cu-BTC powder was also evaluated. The microbreakthrough setup has previously been described elsewhere(44). Briefly, powder was packed approximately 4 mm deep into a 4 mm ID glass fritted tube, while fiber swatches were packed into the tube and compressed to achieve maximum residence time. All samples were evaluated as-received, previously being activated at 170° C. at 1° C./min under vacuum on a Schlenk line. An ammonia feed gas was established from a pressurized ballast mixed with a humidified stream, resulting in a concentration of 1,000 mg/m³ and a relative humidity of 50% at room temperature. The total flow through the glass tube was 20 cc/min. The effluent gas was monitored with a photoionization detector as a function of time. The loading was calculated by integrating effluent curve and subtracting from the challenge concentration, and then dividing by the mass of the sample as detailed previously. (44)

### Pictures of Fabric Samples

Pictures of the fabric samples were taken using a Nikon D600, full frame, SLR, digital camera with a fixed focal length 50 mm AF-S Nikkor 1:1.8 G lens. The camera white balance was calibrated using a white balance cap and the images were captured in .raw format. Pictures are shown without any additional image processing. The .raw files were converted to .tiff file format using Adobe Photoshop CS6.

Shown in FIG. 6 is a selection of samples dyed with gold nanoparticles. FIG. 6a is a sample with 5 nm gold particles on nylon and include a control sample (a-1), nylon dyed with gold after washing with water and chloroform (a-2), and nylon dyed with gold after washing with water, chloroform, soap and water, and acetone (a-3). After washing the gold particles are still present as seen with the purple color of the fabric. FIG. 6b shows a control (b-1), 20 nm gold dyed on cotton after washing with water and chloroform (b-2), and 20 nm gold dyed on cotton after being washed with water, chloroform, soap and water, and acetone (b-3). Likewise, FIG. 6c shows 40 nm gold dyed on cellulose after being washed with water and chloroform (c-2), a control (c-1), and dyeing with gold and subsequent washes with water, chloroform, soap and water, and acetone (c-3). In all cases washing in solvents and soap has very little impact on the gold color of the sample. We note that the nylon control (a-1) has been exposed to gold but was not modified prior to gold exposure with the RDM chemistry. The controls in pictures b and c have not been exposed to gold and have been included to show contrast to the gold dyed samples.

Shown in FIG. 7 are three additional control experiments showing limited color change of cellulose with exposure to gold nanoparticles. Specifically, FIG. 7 is cellulose exposed to 5 nm gold particles overnight followed by washing with water (a), cellulose exposed to 40 nm gold overnight and then washed with water (b), and cellulose exposed to 5 nm gold for 3 nights and subsequently washed with water (c). To ensure washing steps did not promote gold retention (staining) samples of nylon and cellulose were washed in Na₂(CO₃) solution and allowed to sit in gold nanoparticle solution for 1, 2, and 5 days. The results are similar to those shown in FIG. 7 with only limited staining on either nylon or cellulose as shown in FIG. 8.

Specifically, shown in FIG. 8 are nylon samples, washed in Na₂(CO₃) solution as described in the experimental section. Then the samples were exposed to gold for 5, 2, and 1 days shown as samples a, c, and e respectively. Shown as images b, d, and f are samples also washed with Na₂(CO₃) and exposed to gold for 5, 2, and 1 days, respectively, but subsequently washed with water, chloroform, soap, and acetone. For all samples in FIG. 8a-f gold is not appreciably loaded on the fabric.

Likewise, cellulose samples shown as FIG. 8g, i, and k have been washed with Na₂(CO₃) and then exposed to gold for 5, 2, and 1 days respectively. Samples shown as h, j, and l have been washed with Na₂(CO₃) and then washed with water, chloroform, soap and acetone. After washing the fabric gold is not appreciably retained on any of the samples shown in FIG. 8. Although some color is imparted as a stain on the cotton shown in FIG. 8g, this color was only acquired after 5 days of continuous exposure and was not retained after washing as seen in FIG. 8h.

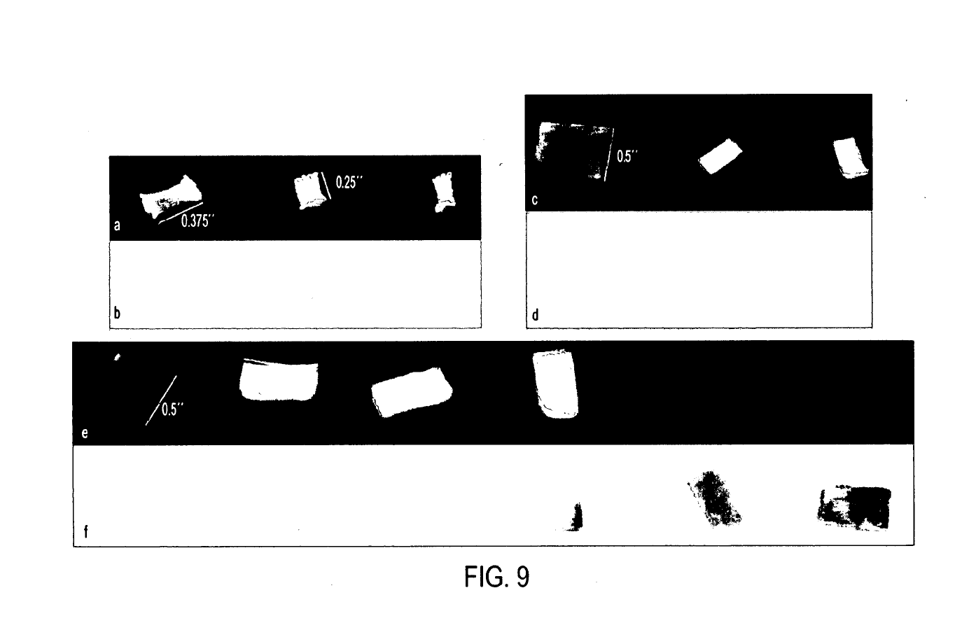

Additional images of the quantum dot modified fabrics are shown in FIG. 9. A fabric sample was modified with cyanuric chloride, functionalized with cysteamine, and then quantum dots were introduced. The quantum dots used in images a-d were purchased as 490 nm dots. To verify the fluorescence of the solution, a drop of the 490 nm quantum dots were placed on a glass slide and the measured fluorescence was 505 nm when using a 405 nm excitation laser. Therefore, the 490 nm quantum dot solution is referred to in the manuscript body as 505 nm quantum dots. The difference between the measured and the wavelength specified by the manufacture is minor; therefore, the other solutions at 525, 575, 630, and 665 nm were not imaged on the confocal microscope to identify any difference between the claimed wavelength and the observed wavelength.

All fabrics, included gold, copper, and quantum dots, were dyed only once with the exception of the cotton fabric swatch shown in FIGS. 9a and b, which was dyed twice. For this sample, the entire RDM process was completed, including soap washings, the fabric was recovered and the RDM process was repeated.

### X-Ray Diffraction Data:

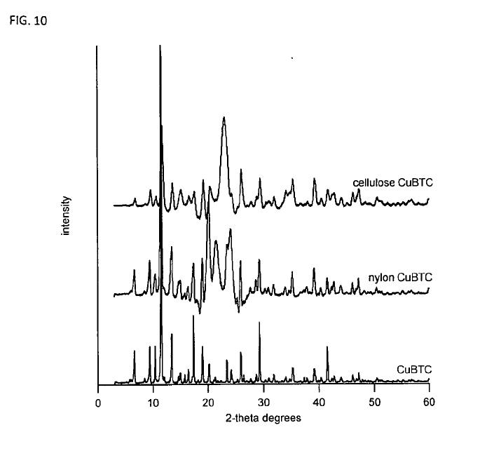

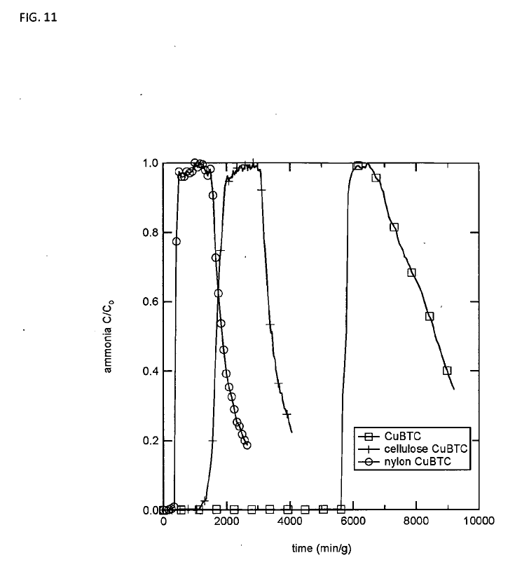

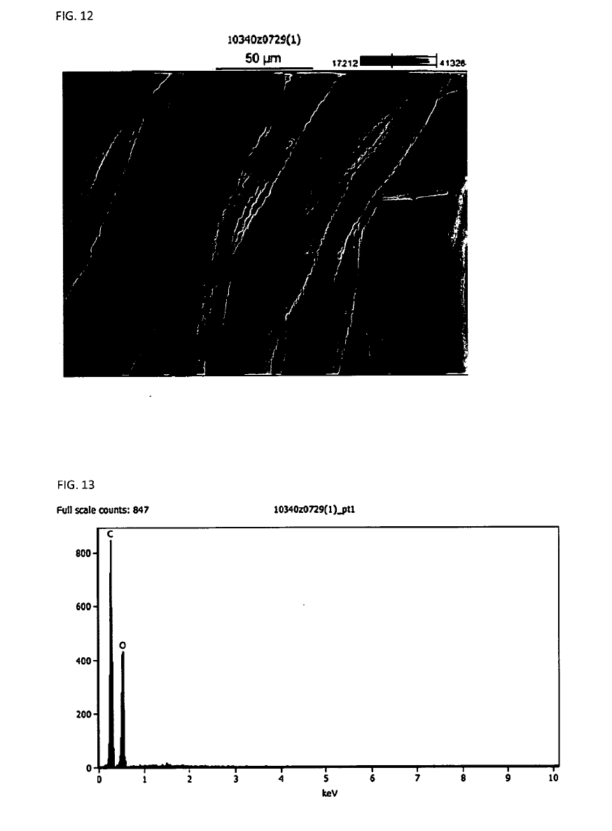

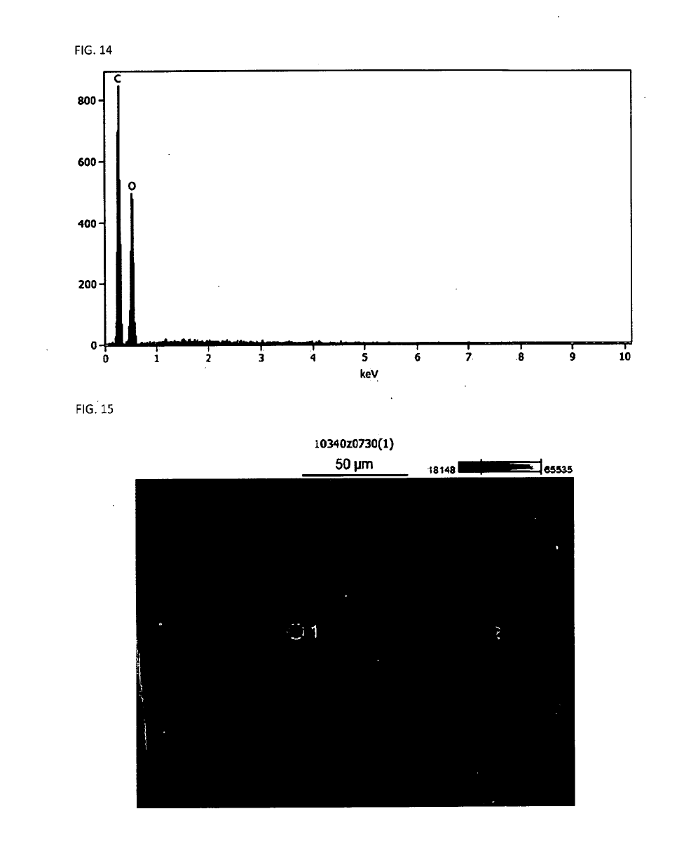

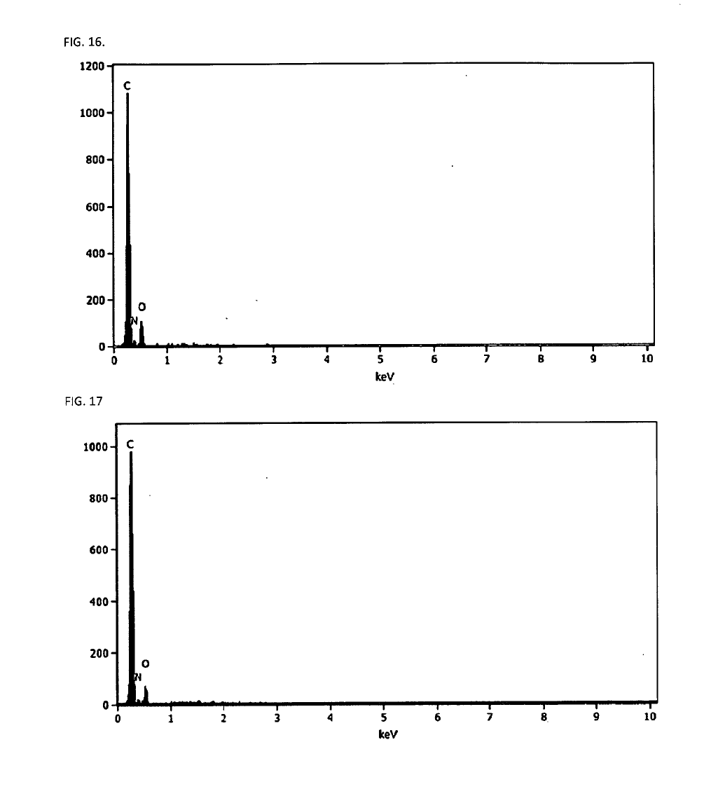

Shown in FIG. 10 are the X-ray diffraction (XRD) patterns for Cu-BTC bound on nylon and cellulose fabric. We note that an additional peak is located at approximately 10.5 2-theta degrees, which is typically not present in simulated XRD patterns. This minor impurity has been seen previously in the literature and is attributed to hydration of the MOF.(48)

Materials synthesis was completed at the University of South Alabama and XRD characterization at the Edgewood Chemical Biological Center (ECBC). The shipping of the samples for XRD characterization may have resulted in this minor impurity as a result of air exposure.

### Nitrogen Isotherm Data for MOF Materials:

Several control experiments were also completed. As described in the synthesis section, the fabric was first treated with the reactive dye chemistry and then exposed to a Cu(NO₃)₂ solution overnight prior to adding the BTC link and beginning the MOF synthesis. During this process, Cu-BTC powder that formed was both attached to the fabric and precipitated from the solution not attached to the fabric. The surface area of the Cu-BTC that collected at the bottom of the synthesis vial unattached to the fabric was collected and the surface area was determined as 1484 and 1763 m²/g for cellulose and nylon respectively. The purpose of this control was to show that the presence of fabric modified with reaction chemistry does not significantly impact the formation of MOF materials. The high surface area of the crystals collected supports this conclusion.

As a control, fabric samples of both nylon and cellulose without RDM chemistry were added to a reaction vial of a traditional Cu-BTC synthesis process. The purpose of this control was to verify that the simple addition of fabric to a MOF synthesis process would not produce fabric with MOF attached on the fabric. The surface area of the cellulose and nylon samples prepared with this method was 326 and 420 m²/g respectively. The lower surface area of these materials shows that the reactive dye chemistry allows for more MOF material to be added to the fabric, as seen with a higher surface area than this control group.

During the RDM the reactive dye modified fabric sample was exposed to Cu(NO₃)₂ overnight prior to beginning MOF synthesis. Therefore, to determine if diffusion of Cu into the fibers of the material was simply time limited during a typical Cu-BTC synthesis, nylon and cellulose materials were exposed to Cu(NO₃)₂ overnight without reactive dye attachment chemistry. After exposure to Cu overnight, the MOF synthesis procedure was started. In this control, exposing the fabric to Cu for a longer period of time resulted in a surface area of 419 m²/g for cellulose. However, when this process was repeated and the sample was then washed with solvents, the surface area was 95 m²/g, which is significantly lower than without washing indicating a large portion of the MOF was physically attached to the material and not chemically bound to the fabric surface. In either case, with or without washing, both controls produced areas that are lower than the reactive dye chemistry based samples.

The results of each of these control runs are summarized in Tables 1-3. The Cu-BTC sample used to produce the ethane and ethylene isotherms shown in FIG. 3e was not activated as aggressively as the other Cu-BTC samples and had a surface area of 671 m²/g.

TABLE 1
Summary of BET surface area measurements of Cu-BTC powders.

| BET (m²/g) | Sample |
|---|---|
| 1778 | Pure Cu-BTC - No Fabric in Solution |
| 1484 | Cu-BTC formed in solution with Cotton Sample |
| 1763 | Cu-BTC formed in solution with Nylon Sample |

TABLE 2
BET surface area measurements of control experiments of
Cu-BTC and Nylon

| BET | |
|---|---|
| (m²/g) | Sample |
| 680 | Nylon - Cu-BTC- with reaction, Cu(NO₃)₂ exposure for one |
| | day, no rigorous washing |
| 420 | Nylon - Cu-BTC Control - no reaction, no rigorous washing |
| 95 | Nylon - Cu-BTC Control - no rxn, Cu(NO₃)₂ exposed for one |
| | day, with rigorous washing |
| 196 | Nylon - Cu-BTC- with reaction, Cu(NO₃)₂ exposure for one |
| | day, with rigorous washing |

TABLE 3
BET surface area measurements of control experiments of
Cu-BTC and cotton

| BET | |
|---|---|
| (m²/g) | Sample |
| 671 | Cotton - Cu-BTC- with reaction, Cu(NO₃)₂ exposure for one |
| | day, no rigorous washing |
| 326 | Cotton - Cu-BTC Control - no reaction, no rigorous washing |
| 419 | Cotton - Cu-BTC Control - no reaction, Cu(NO₃)₂ exposure |
| | for one day, with rigorous washing |
| 976 | Cotton- Cu-BTC- with reaction, Cu(NO₃)₂ exposure for one |
| | day, with rigorous washing |

### Ammonia Breakthrough Experiments:

Results of the microbreakthrough experiments are shown in FIG. 11. The results of the breakthrough experiments are consistent with previous ammonia breakthrough experiments (47). Specifically, the previous report notes a loading of 5.4 mol/kg for Cu-BTC from an air stream at 80% relative humidity (RH) at 20° C. and a feed concentration of 1000 mg/m³. The Cu-BTC control in the current work produced a loading of 6.75 mol/kg at 50% RH at 20° C. For the current report, the samples were run as received and were not preconditioned in air matching the RH of the experiment.

The fabric samples each showed lower loadings of ammonia than the pure Cu-BTC sample, which is consistent with the lower surface area of the fabrics relative to the pure Cu-BTC. Also, the performance of the fabric swatches is impacted by the weight of the inactive cellulose or nylon fiber substrate. The cellulose sample produced a loading of 1.97 mol/kg and the nylon 0.49 mol/kg. Desorption was not calculated.

### REFERENCES AND NOTES

1. A. P. S. Sawhney, B. Condon, K. V. Singh, S. S. Pang, G. Li, et al., Textile Research Journal 78, 731 (2008). 2. J. He, T. Kunitake, A. Nakao, Chem. Mater. 15, 4401 (2003). 3. Z.-D. Qi, T. Saito, Y. Fan, A. Isogai, Biomacromolecules 13, 553 (2012). 4. H. Dong, J. P. Hinestroza, App. Mater. Inter. 1, 797 (2009). 5. K. Hyde, M. Rusa, J. Hinestroza, Nanotechnology 16, 422 (2005). 6. K. Hyde, H. Dong, J. P. Hinestroza, Cellulose 14, 615 (2007). 7. S. T. Dubas, P. Kumlangdudsana, P. Potiyaraj, Colloids and Surfaces A: Physicochem. Eng. Aspects 289, 105 (2006). 8. J. Gao, B. Zhao, M. E. Itkis, E. Bekyarova, H. Hu, et al., J. Amer. Chem. Soc. 128, 7492 (2006). 9. Zhao, J., Losego, M. D., Lemaire, P. C., Williams, P. S., Gong, B., Atanasov, S. E., Blevins, T. M., Oldham, C. J., Walls, H. J., Shepherd, S. D., Browe, M. A., Peterson, G. W., Parsons, G. N. (2014). Highly Adsorptive, MOF-Functionalized Nonwoven Fiber Mats for Hazardous Gas Capture Enabled by Atomic Layer Deposition. Adv. Mater. Interfaces, 1: 1400040. doi: 10.1002/admi.201400040. 10. F. J. Xu, J. P. Zha, E. T. Kang, K. G. Neoh, J. Li, Langmuir 23, 8585 (2007). 11. M. Meilikhov, K. Yusenko, E. Schollmeyer, C. Mayer, H. Buschmann, et al., Dalton Trans. 40, 4838 (2011). 12. J. Cai, S. Kimura, M. Wada, S. Kuga, Biomacromolecules 10, 87 (2009). 13. L. J. Castellanos, C. Blanco-Tirado, J. P. Hinestroza, M. Y. Combariza, Cellulose 19, 1933 (2012). 14. J. Song, N. L. Birbach, J. P. Hinestroza, Cellulose 19, 411 (2012). 15. S. Y. Park, J. W. Chung, R. D. Priestly, S.-Y. Kwak, Cellulose 19, 2141 (2012). 16. S. Yokota, T. Kitaoka, M. Opietnik, T. Rosenau, H. Wariishi, Angew. Chem. Int. Ed. 47, 9866 (2008). 17. G. Mary, S. K. Bajpai, N. Chand, Journal of Applied Polymer Science 113, 757 (2009). 18. A. Yadav, V. Prasad, A. A. Kathe, S. Raj, D. Yadav, et al., Bull. Mater. Sci. 29, 641 (2006). 19. B. Jia, Y. Mei, L. Cheng, J. Zhou, L. Zhang, ACS Appl. Mater. Interfaces 4, 2897 (2012). 20. H. H. A. Dollwet, J. R. J. Sorenson, Trace Elem. Med. Bio. 2, 80 (1985). 21. C. Zhu, J. Xue, J. He, J. Nanosci. Nanotechnol. 9, 3067 (2009). 22. S. Wei, J. Sampathi, Z. Guo, N. Anumandla, D. Rutman, et al., Polymer 52, 5817 (2011). 23. Y. Yuan, F.-S. Riehle, R. Nitschke, M. Krüger, Materials Science and Engineering B 177, 245 (2012). 24. A. C. Small, J. H. Johnston, N. Clark, Eur. J. Inorg. Chem. 242 (2010). 25. R. Ostermann, J. Cravillon, C. Weidmann, M. Wiebcke, B. M. Smarsly, Chem. Commun. 47, 442 (2011). 26. Y. Wu, F. Li, H. Liu, W. Zhu, M. Teng, et al., J. Mater. Chem. 22, 16971 (2012). 27. P. Kuesgens, S. Siegle, S. Kaskel, Adv. Eng. Mater. 11, 93 (2009). 28. M. da Silca Pinto, C. A. Sierra-Avila, J. P. Hinestroza, Cellulose 19, 1771 (2012). 29. M. Meilikhov, K. Yusenko, E. Schollmeyer, C. Meyer, H.-J. Buschmann, et al., Dalton Trans. 40, 4838 (2011). 30. A. R. Abbasi, K. Akhbari, A. Morsali, Ultrason. Sonochem. 19, 846 (2012). 31. A. Centrone, Y. Yang, S. Speakman, L. Bromberg, G. C. Rutledge, et al., J. Am. Chem. Soc. 132, 15687 (2010). 32. J.-L. Zhuang, D. Ar, X.-J. Yu, J.-X. Liu, A. Terfort, Adv. Mater. 25, 4631 (2013). 33. Handbook of Textile and Industrial Dyeing Vol 1: Principles, processes and types of dyes. Ed. M. Clark, Woodhead, Cambridge, 2011. 34. Analysis, Synthesis and Design of Chemical Processes, 4th Ed., R. Turton, R. C. Bailie, W. B. Whiting, J. A. Shaeiwitz, D. Bhattacharyya Prentice Hall PTR, Upper Saddle River, New Jersey 07458, 2012. 35. A. H. M. Renfrew, J. A. Taylor, Rev. Prog. Coloration 20, 1 (1990). 36. A. Soleimani-Gorgani, J. A. Taylor, Dyes and Pigments 68, 119 (2006). 37. H. J. Cho, D. M. Lewis, Coloration Technology 118, 198 (2002). 38. A. Soleimani-Gorgani, J. A. Taylor, Dyes and Pigments 68, 109 (2006). 39. S. M. Burkinshaw, A. E. Wills, Dyes and Pigments 34, 243 (1997). 40. W. Zuwang, Rev. Prog. Coloration 28, 32 (1998). 41. S. Underwood, P. Mulvaney, Langmuir 10, 3427 (1994). 42. M. Rungta, C. Zhang, W. J. Koros, L. Xu, AIChE 59, 3475 (2013). 43. Q. M. Wang, D. Shen, M. Bulow, M. L. Lau, S. Deng, F. R. Fitch, N. O. Lemcoff, J. Semanscin, Micro. Meso. Mater. 55, 217 (2002). 44. J. B. Decoste, G. W. Peterson, M. W. Smith, C. A. Stone, C. R. Willis, J. Am. Chem. Soc. 134, 1486 (2012). 45. Q. M. Wang, D. Shen, M. Bulow, M. L. Lau, S. Deng, Micro. Meso. Mater. 55, 217 (2002). 46. T. G. Glover, G. W. Peterson, B. J. Schindler, D. Britt, O. Yaghi, Chem. Eng. Sci. 66, 163 (2011). 47. G. W. Peterson, G. W. Wagner, A. Balboa, J. Mahle, T. Sewell, et al., J. Phys. Chem. C 113, 13906 (2009). 48. K. Schlichte, T. Kratzke, S. Kaskel, Micro. Meso. Mater. 73, 81 (2004).

### F. Conclusions

It is to be understood that any given elements of the disclosed embodiments of the invention may be embodied in a single structure, a single step, a single substance, or the like. Similarly, a given element of the disclosed embodiment may be embodied in multiple structures, steps, substances, or the like.

The foregoing description illustrates and describes the processes, machines, manufactures, compositions of matter, and other teachings of the present disclosure. Additionally, the disclosure shows and describes only certain embodiments of the processes, machines, manufactures, compositions of matter, and other teachings disclosed, but, as mentioned above, it is to be understood that the teachings of the present disclosure are capable of use in various other combinations, modifications, and environments and are capable of changes or modifications within the scope of the teachings as expressed herein, commensurate with the skill and/or knowledge of a person having ordinary skill in the relevant art. The embodiments described hereinabove are further intended to explain certain best modes known of practicing the processes, machines, manufactures, compositions of matter, and other teachings of the present disclosure and to enable others skilled in the art to utilize the teachings of the present disclosure in such, or other, embodiments and with the various modifications required by the particular applications or uses. Accordingly, the processes, machines, manufactures, compositions of matter, and other teachings of the present disclosure are not intended to limit the exact embodiments and examples disclosed herein. Any section headings herein are provided only for consistency with the suggestions of 37 C.F.R. §1.77 or otherwise to provide organizational queues. These headings shall not limit or characterize the invention(s) set forth herein.

1. (canceled)

2. (canceled)

3. (canceled)

4. (canceled)

5. A method of attaching a functional nanostructure to a fiber substrate using a reactive dye conjugating moiety, the method comprising the following steps in any order: (a) covalently binding the reactive dye conjugating moiety to the fiber substrate; (b) covalently binding a bonding agent to the reactive dye conjugating moiety; and (c) binding the functional nanostructure to the bonding agent; wherein the functional nanostructure is a metal-organic framework (MOF), wherein step (c) comprises binding a metal ion to the bonding agent, and wherein the method comprises conjugating the metal ion to an organic linker.

6. The method of claim 5 comprising performing step (a) prior to steps (b) and (c), and performing step (b) prior to step (c).

7. (canceled)

8. (canceled)

9. The method of claim 5, said bonding agent comprising a thiol group bound to the functional nanostructure and a primary amine group bound to the reactive dye conjugating moiety.

10. The method of claim 5, said bonding agent having the formula H₂N—R—SH, wherein R is a substituted or unsubstituted alkyl group.

11. (canceled)

12. (canceled)

13. (canceled)

14. (canceled)

15. The method of claim 10, said bonding agent having the formula H₂N—R—SH, wherein R is (CH₂)ₙ, in which n=1-60.

16. (canceled)

17. (canceled)

18. (canceled)

19. (canceled)

20. The method of claim 15, said bonding agent having the formula H₂N—R—SH, wherein R is (CH₂)ₙ, in which n is either 3 or 6.

21. The method of claim 5, wherein the bonding agent is one of cysteamine and cysteamine hydrochloride.

22. (canceled)

23. (canceled)

24. (canceled)

25. (canceled)

26. The method of claim 5, in which the functional nanostructure is a copper-1,3,5-benzenetricarboxylic acid framework.

27. (canceled)

28. (canceled)

29. (canceled)

30. The method of claim 5, wherein the reactive dye conjugating moiety comprises at least two reactive binding groups, each independently selected from the group consisting of: chloride, fluoride, thiol, sulfide, sulfone, and amide.

31. The method of claim 5, wherein the reactive dye conjugating moiety is a chlorotriazine dye.

32. (canceled)

33. The method of claim 5, wherein the reactive dye conjugating moiety is selected from the group consisting of: cyanuric chloride, monochlorotriazine, monofluorochlorotriazine, dichlorotriazine, monofluorodichlorotriazine, vinyl sulfone and difluoromonochlorotriazine.

34. The method of claim 5, wherein the functional nanostructure is a gold nanoparticle, and further comprising a functionalized thiol bound to the gold nanoparticle.

35. (canceled)

36. The method of claim 5, wherein the functional nanostructure is a gold nanoparticle, and further comprising a functionalized thiol covalently bound to the gold nanoparticle, said functionalized thiol comprising a sulfide moiety conjugated to a second functional nanostructure selected from the group consisting of: a self-assembled monolayer forming compound, gold, silver, metal nanoparticles, bulk metal particles, metal salts, metalloids, metal alloys, quantum dots, adsorbents, catalysts, metal oxides, magnetic nanoparticles, polyoxometlataes (POMS), polymers, polymers of intrinsic microporosity (PIMS), metal organic frameworks (MOFs), an organic linker of a MOF, zeolites, covalent organic frameworks (COFs), zeolites, zeolitic imidazolate frameworks (ZIFs), graphene oxide frameworks (GOFs), colloidal particles, silicas, carbons, graphene, graphite, antibiotics, antibodies, fluorescent molecules, enzymes, and proteins.

37. (canceled)

38. (canceled)

39. The method of claim 5, in which the fiber substrate is a polyamide fiber.

40. (canceled)

41. (canceled)

42. (canceled)

43. The method of claim 5, in which the fiber substrate is a cellulosic fiber.

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. A coated fiber coated with a functional nanostructure that is the product of the method of claim 5.

61. The coated fiber of claim 60, wherein no significant loss of the functional nanostructure occurs upon vigorous washing.

62. (canceled)

63. The coated fiber of claim 60, wherein no significant loss of the functional nanostructure occurs upon vigorous washing with all of: chloroform, water, soap and water, tetrahydrofuran, hexane, and acetone.

64. A method of attaching a functional nanostructure to a fiber substrate using a reactive dye conjugating moiety, the method comprising the following steps in any order: (a) covalently binding the reactive dye conjugating moiety to the fiber substrate; (b) covalently binding a bonding agent to the reactive dye conjugating moiety; and (c) binding the functional nanostructure to the bonding agent, wherein the reactive dye conjugating moiety is a chlorotriazine moiety; wherein the bonding agent is cysteamine or cysteamine hydrochloride; and wherein the functional nanostructure is selected from the group consisting of: a gold nanoparticle of less than about 50 nm diameter, a metal-organic framework, and a quantum dot.

65. A cotton textile comprising the coated fiber of claim 60.
